(19) United States
(12) Patent Application Publication
(10) Pub. No.: US 2025/0190949 A1
(43) Pub. Date: Jun. 12, 2025

(54) FACILITATING PERSONAL DEVELOPMENT OF AN INDIVIDUAL

(71) Applicant: Futurecite Inc., Edmonton (CA)

(72) Inventors: Yuan Tian, Ontario (CA); Eva Borsato, Edmonton (CA)

(73) Assignee: Futurecite Inc., Edmonton, AB (CA)

(21) Appl. No.: 18/842,330

(22) PCT Filed: Feb. 28, 2023
(86) PCT No.: PCT/CA2023/050260

§ 371 Date: Aug. 28, 2024

Related U.S. Application Data
(60) Provisional application No. 63/315,016, filed on Feb. 28, 2022.

(51) Int. Cl. G06Q 10/1053; G06Q 50/20; G16H 50/30

(52) U.S. Cl. CPC G06Q 10/1053; G06Q 50/205; G16H 50/30

(57) ABSTRACT

According to at least one embodiment, there is disclosed a method of matching an individual to at least one opportunity. The method involves causing at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

### CROSS-REFERENCE TO RELATED APPLICATION

This application claims priority to U.S. provisional patent application No. 63/315,016 filed on Feb. 28, 2022. In the United States, this application is also a continuation-in-part of International patent application no. PCT/CA2021/051207 filed on Aug. 31, 2021, which claims priority to Canadian patent application no. 3091768 filed on Sep. 1, 2020. The entire contents of U.S. provisional patent application No. 63/315,016, International patent application no. PCT/CA2021/051207, and Canadian patent application no. 3091768 are incorporated by reference herein.

### FIELD

This disclosure relates generally to facilitating personal development of an individual, for example by matching the individual to one or more opportunities based one or more inferred personality characteristics of the individual.

### RELATED ART

Effectively matching an individual to an appropriate opportunity can be important, particularly in modern economies. However, existing methods and systems of matching individuals to opportunities may not sufficiently account for personality characteristics of an individual.

### SUMMARY

According to at least one embodiment, there is disclosed a method of facilitating personal development of an individual, the method comprising causing at least one processor circuit to, at least, in response to a match between the individual and at least one opportunity, the at least one opportunity withheld from the individual prior to the match, present the at least one opportunity to the individual.

In some embodiments, the method further comprises causing the at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

According to at least one embodiment a method of matching an individual to at least one opportunity, the method comprising causing at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

In some embodiments, the at least one personality characteristic comprises a personality type.

In some embodiments, the at least one personality characteristic comprises a personality trait.

In some embodiments, the at least one personality characteristic comprises a personality state.

In some embodiments, the at least one opportunity comprises at least one learning opportunity.

In some embodiments, the at least one opportunity comprises at least one employment opportunity.

In some embodiments, the at least one opportunity comprises at least one employment opportunity and at least one learning opportunity providing knowledge associated with the at least one employment opportunity.

In some embodiments, the at least one learning opportunity comprises at least one remote learning opportunity.

In some embodiments, the inference is further based on at least one measurement of a body of the individual.

In some embodiments, the method further comprises causing the at least one processor circuit to, at least, receive, from at least one measurement device, at least one signal representing the at least one measurement.

In some embodiments, the method further comprises causing the at least one processor circuit to, at least, cause the at least one measurement device to measure the at least one measurement.

In some embodiments, the at least one measurement device is worn on the body of the individual when the at least one measurement device measures the at least one measurement.

In some embodiments, the at least one measurement device comprises at least one haptic measurement device.

In some embodiments, the at least one measurement device comprises at least one smartwatch.

In some embodiments, the at least one measurement device comprises at least one fitness tracker.

In some embodiments, the at least one measurement device comprises at least one heart rate monitor.

In some embodiments, the at least one measurement device comprises at least one sleep monitor.

In some embodiments, the at least one measurement device comprises at least one virtual reality headset.

In some embodiments, the at least one measurement device comprises smart glasses.

In some embodiments, the at least one measurement device comprises smart clothing.

In some embodiments, the at least one measurement device comprises smart jewelry.

In some embodiments, the at least one measurement device comprises smart shoes.

In some embodiments, the at least one measurement device comprises a smart hat.

In some embodiments, the at least one measurement comprises at least one haptic measurement of the body of the individual.

In some embodiments, the at least one measurement comprises at least one biometric measurement of the body of the individual.

In some embodiments, the at least one biometric measurement comprises a measurement of heart rate.

In some embodiments, the at least one biometric measurement comprises a measurement of body temperature.

In some embodiments, the at least one biometric measurement comprises a measurement of respiratory rate.

In some embodiments, the at least one biometric measurement comprises a measurement of blood pressure.

In some embodiments, the at least one biometric measurement comprises a measurement of oxygen saturation.

In some embodiments, the at least one biometric measurement comprises a measurement of electrodermal activity.

In some embodiments, the at least one biometric measurement comprises a measurement of movement.

In some embodiments, the at least one biometric measurement comprises a measurement of posture.

In some embodiments, the at least one biometric measurement comprises a measurement of sleep patterns.

In some embodiments, the at least one biometric measurement comprises a measurement of nutrition intake.

In some embodiments, the at least one biometric measurement comprises a measurement of environmental exposure.

In some embodiments, the measurement of environmental exposure comprises a measurement of temperature.

In some embodiments, the measurement of environmental exposure comprises a measurement of humidity.

In some embodiments, the measurement of environmental exposure comprises a measurement of air quality.

In some embodiments, the at least one measurement comprises at least one geolocation measurement of the body of the individual.

In some embodiments, the method further comprises causing the at least one processor circuit to, at least, infer, from the at least one response of the individual and the at least one measurement of the body of the individual, the at least one personality characteristic of the individual.

In some embodiments, causing the at least one processor circuit to infer the at least one personality characteristic of the individual comprises causing the at least one processor circuit to corroborate the at least one response of the individual using the at least one measurement of the body of the individual.

In some embodiments, the method further comprises causing the at least one processor circuit to, at least, infer, from the at least one response of the individual, the at least one personality characteristic of the individual.

In some embodiments, the at least one response indicates at least one interest of the individual; and causing the at least one processor circuit to infer the at least one personality characteristic of the individual comprises causing the at least one processor circuit to infer the at least one personality characteristic of the individual from the at least one interest of the individual.

In some embodiments, causing the at least one processor circuit to infer the at least one personality characteristic of the individual comprises: for each personality characteristic of a plurality of personality characteristics, causing the at least one processor circuit to calculate a respective probability that the individual belongs to the personality characteristic; and causing the at least one processor circuit to rank the plurality of personality characteristics according to the respective probabilities.

In some embodiments, the plurality of personality characteristics comprises the Holland Codes.

In some embodiments, the method further comprises causing the at least one processor circuit to, at least, receive at least one incoming signal comprising the at least one response.

In some embodiments, causing the at least one processor circuit to receive the at least one incoming signal comprises causing the at least one processor circuit to receive the at least one incoming signal from an input device.

In some embodiments, the method further comprises causing the at least one processor circuit to, at least, send at least one outgoing signal comprising the at least one prompt.

In some embodiments, causing the at least one processor circuit to send the at least one outgoing signal comprises causing the at least one processor circuit to send the at least one outgoing signal to an output device.

In some embodiments, the method further comprises causing the at least one processor circuit to, at least, generate the at least one prompt.

In some embodiments, causing the at least one processor circuit to generate the at least one prompt comprises causing the at least one processor circuit to dynamically generate the at least one prompt.

In some embodiments, the at least one processor circuit dynamically generates the at least one prompt using, at least, AI-generated media.

In some embodiments, the at least one processor circuit dynamically generates the at least one prompt using, at least, a large language model.

In some embodiments, the at least one processor circuit dynamically generates the at least one prompt using, at least, a text-to-image model.

In some embodiments, the at least one processor circuit dynamically generates the at least one prompt using, at least, a text-to-speech model.

In some embodiments, the at least one processor circuit dynamically generates the at least one prompt using, at least, a text-to-video model.

In some embodiments, the at least one processor circuit dynamically generates the at least one prompt using, at least, an AI-based code completion tool.

In some embodiments, the at least one response comprises a plurality of responses; the at least one prompt comprises a plurality of prompts; and each response of the plurality of responses is responsive to a respective at least one prompt of the plurality of prompts.

In some embodiments, the plurality of responses comprises at least five responses.

In some embodiments, the plurality of prompts comprises at least five prompts.

In some embodiments, causing the at least one processor circuit to match the individual to the at least one opportunity based on the inferred at least one personality characteristic of the individual comprises causing the at least one processor circuit to compare the inferred at least one personality characteristic to at least one preferred personality characteristic aligned with the opportunity.

In some embodiments, causing the at least one processor circuit to compare the inferred at least one personality characteristic of the individual to the at least one preferred personality characteristic aligned with the opportunity comprises causing the at least one processor circuit to calculate at least one matching score representing a pairing of the inferred at least one personality characteristic with the at least one preferred personality characteristic.

In some embodiments, causing the at least one processor circuit to compare the inferred at least one personality characteristic of the individual to the at least one preferred personality characteristic aligned with the opportunity comprises, for each posting of a plurality of postings comprising the opportunity, causing the at least one processor circuit to calculate a respective matching score representing a pairing of the inferred at least one personality characteristic with at least one preferred personality characteristic aligned with the posting.

In some embodiments, causing the at least one processor circuit to compare the inferred at least one personality characteristic of the individual to the at least one preferred personality characteristic aligned with the opportunity comprises, for each candidate of a plurality of candidates comprising the individual, causing the at least one processor circuit to calculate a respective matching score representing a pairing of at least one personality characteristic of the candidate with the at least one preferred personality characteristic.

In some embodiments, causing the at least one processor circuit to compare the inferred at least one personality characteristic of the individual to the at least one preferred personality characteristic aligned with the opportunity comprises, for each posting of a plurality of postings comprising the opportunity, for each candidate of a plurality of candidates comprising the individual, causing the at least one processor circuit to calculate a respective matching score representing a pairing of at least one personality characteristic of the candidate with at least one preferred personality characteristic aligned with the posting.

In some embodiments, the at least one preferred personality characteristic comprises a plurality of preferred personality characteristics ranked in order of preference.

In some embodiments, causing the at least one processor circuit to match the individual to the at least one opportunity comprises causing the at least one processor circuit to match the individual to the at least one opportunity further based at least on at least one skill of the individual.

According to at least one embodiment, there is disclosed a system for facilitating personal development of an individual, the system comprising: at least one processor circuit; and at least one computer-readable storage medium comprising stored thereon program codes that, when executed by the at least one processor circuit, cause the at least one processor circuit to, at least, in response to a match between the individual and at least one opportunity, the at least one opportunity withheld from the individual prior to the match, present the at least one opportunity to the individual.

In some embodiments, the program codes, when executed by the at least one processor circuit, further cause the at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

According to at least one embodiment, there is disclosed a system for matching an individual to at least one opportunity, the system comprising: at least one processor circuit; and at least one computer-readable storage medium comprising stored thereon program codes that, when executed by the at least one processor circuit, cause the at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

In some embodiments, the at least one personality characteristic comprises a personality type.

In some embodiments, the at least one personality characteristic comprises a personality trait.

In some embodiments, the at least one personality characteristic comprises a personality state.

In some embodiments, the at least one opportunity comprises at least one learning opportunity.

In some embodiments, the at least one opportunity comprises at least one employment opportunity.

In some embodiments, the at least one opportunity comprises at least one employment opportunity and at least one learning opportunity providing knowledge associated with the at least one employment opportunity.

In some embodiments, the at least one learning opportunity comprises at least one remote learning opportunity.

In some embodiments, the inference is further based on at least one measurement of a body of the individual.

In some embodiments, the program codes, when executed by the at least one processor circuit, further cause the at least one processor circuit to, at least, receive, from at least one measurement device, at least one signal representing the at least one measurement.

In some embodiments, the program codes, when executed by the at least one processor circuit, further cause the at least one processor circuit to, at least, cause the at least one measurement device to measure the at least one measurement.

In some embodiments, the system further comprises the at least one measurement device.

In some embodiments, the at least one measurement device is wearable on the body of the individual when the at least one measurement device measures the at least one measurement.

In some embodiments, the at least one measurement device comprises at least one haptic measurement device.

In some embodiments, the at least one measurement device comprises at least one smartwatch.

In some embodiments, the at least one measurement device comprises at least one fitness tracker.

In some embodiments, the at least one measurement device comprises at least one heart rate monitor.

In some embodiments, the at least one measurement device comprises at least one sleep monitor.

In some embodiments, the at least one measurement device comprises at least one virtual reality headset.

In some embodiments, the at least one measurement device comprises smart glasses.

In some embodiments, the at least one measurement device comprises smart clothing.

In some embodiments, the at least one measurement device comprises smart jewelry.

In some embodiments, the at least one measurement device comprises smart shoes.

In some embodiments, the at least one measurement device comprises a smart hat.

In some embodiments, the at least one measurement comprises at least one haptic measurement of the body of the individual.

In some embodiments, the at least one measurement comprises at least one biometric measurement of the body of the individual.

In some embodiments, the at least one measurement comprises a measurement of heart rate.

In some embodiments, the at least one measurement comprises a measurement of body temperature.

In some embodiments, the at least one measurement comprises a measurement of respiratory rate.

In some embodiments, the at least one measurement comprises a measurement of blood pressure.

In some embodiments, the at least one measurement comprises a measurement of oxygen saturation.

In some embodiments, the at least one measurement comprises a measurement of electrodermal activity.

In some embodiments, the at least one measurement comprises a measurement of movement.

In some embodiments, the at least one measurement comprises a measurement of posture.

In some embodiments, the at least one measurement comprises a measurement of sleep patterns.

In some embodiments, the at least one measurement comprises a measurement of nutrition intake.

In some embodiments, the at least one measurement comprises a measurement of environmental exposure.

In some embodiments, the measurement of environmental exposure comprises a measurement of temperature.

In some embodiments, the measurement of environmental exposure comprises a measurement of humidity.

In some embodiments, the measurement of environmental exposure comprises a measurement of air quality.

In some embodiments, the at least one measurement comprises at least one geolocation measurement of the body of the individual.

In some embodiments, the program codes, when executed by the at least one processor circuit, further cause the at least one processor circuit to, at least, infer, from the at least one response of the individual and the at least one measurement of the body of the individual, the at least one personality characteristic of the individual.

In some embodiments, the program codes, when executed by the at least one processor circuit, cause the at least one processor circuit to corroborate the at least one response of the individual using the at least one measurement of the body of the individual.

In some embodiments, the program codes, when executed by the at least one processor circuit, further cause the at least one processor circuit to, at least, infer, from the at least one response of the individual, the at least one personality characteristic of the individual.

In some embodiments, the at least one response indicates at least one interest of the individual; and the program codes, when executed by the at least one processor circuit, cause the at least one processor circuit to infer the at least one personality characteristic of the individual from the at least one interest of the individual.

In some embodiments, for each personality characteristic of a plurality of personality characteristics, the program codes, when executed by the at least one processor circuit, cause the at least one processor circuit to calculate a respective probability that the individual belongs to the personality characteristic; and the program codes, when executed by the at least one processor circuit, cause the at least one processor circuit to rank the plurality of personality characteristics according to the respective probabilities.

In some embodiments, the plurality of personality characteristics comprises the Holland Codes.

In some embodiments, the program codes, when executed by the at least one processor circuit, further cause the at least one processor circuit to, at least, receive at least one incoming signal comprising the at least one response.

In some embodiments, the program codes, when executed by the at least one processor circuit, further cause the at least one processor circuit to receive the at least one incoming signal from an input device.

In some embodiments, the program codes, when executed by the at least one processor circuit, further cause the at least one processor circuit to, at least, send at least one outgoing signal comprising the at least one prompt.

In some embodiments, the program codes, when executed by the at least one processor circuit, further cause the at least one processor circuit to send the at least one outgoing signal to an output device.

In some embodiments, the program codes, when executed by the at least one processor circuit, further cause the at least one processor circuit to, at least, generate the at least one prompt.

In some embodiments, the program codes, when executed by the at least one processor circuit, cause the at least one processor circuit to dynamically generate the at least one prompt.

In some embodiments, the at least one processor circuit dynamically generates the at least one prompt using, at least, AI-generated media.

In some embodiments, the at least one processor circuit dynamically generates the at least one prompt using, at least, a large language model.

In some embodiments, the at least one processor circuit dynamically generates the at least one prompt using, at least, a text-to-image model.

In some embodiments, the at least one processor circuit dynamically generates the at least one prompt using, at least, a text-to-speech model.

In some embodiments, the at least one processor circuit dynamically generates the at least one prompt using, at least, a text-to-video model.

In some embodiments, the at least one processor circuit dynamically generates the at least one prompt using, at least, an AI-based code completion tool.

In some embodiments, the at least one response comprises a plurality of responses; the at least one prompt comprises a plurality of prompts; and each response of the plurality of responses is responsive to a respective at least one prompt of the plurality of prompts.

In some embodiments, the plurality of responses comprises at least five responses.

In some embodiments, the plurality of prompts comprises at least five prompts.

In some embodiments, the program codes, when executed by the at least one processor circuit, cause the at least one processor circuit to compare the inferred at least one personality characteristic to at least one preferred personality characteristic aligned with the opportunity.

In some embodiments, the program codes, when executed by the at least one processor circuit, cause the at least one processor circuit to calculate at least one matching score representing a pairing of the inferred at least one personality characteristic with the at least one preferred personality characteristic.

In some embodiments, for each posting of a plurality of postings comprising the opportunity, the program codes, when executed by the at least one processor circuit, cause the at least one processor circuit to calculate a respective matching score representing a pairing of the inferred at least one personality characteristic with at least one preferred personality characteristic aligned with the posting.

In some embodiments, for each candidate of a plurality of candidates comprising the individual, the program codes, when executed by the at least one processor circuit, cause the at least one processor circuit to calculate a respective matching score representing a pairing of at least one personality characteristic of the candidate with the at least one preferred personality characteristic.

In some embodiments, for each posting of a plurality of postings comprising the opportunity, for each candidate of a plurality of candidates comprising the individual, the program codes, when executed by the at least one processor circuit, cause the at least one processor circuit to calculate a respective matching score representing a pairing of at least one personality characteristic of the candidate with at least one preferred personality characteristic aligned with the posting.

In some embodiments, the at least one preferred personality characteristic comprises a plurality of preferred personality characteristics ranked in order of preference.

In some embodiments, the program codes, when executed by the at least one processor circuit, cause the at least one processor circuit to match the individual to the at least one opportunity further based at least on at least one skill of the individual.

Other aspects and features will become apparent to those ordinarily skilled in the art upon review of the following description of illustrative embodiments in conjunction with the accompanying figures.

### BRIEF DESCRIPTION OF THE DRAWINGS

FIG. 1 illustrates a system for matching an individual to one or more opportunities according to one embodiment.

FIG. 2 illustrates a processor circuit of a server computing device of the system of FIG. 1.

FIG. 3 illustrates a prompts table entry of a prompts store of the processor circuit of FIG. 2

FIG. 4 illustrates a multi-mode data processing system according to another embodiment.

FIG. 5 illustrates examples of possible measurement devices according to at least some embodiments as described herein.

FIG. 6 illustrates a federated learning model of the data processing system of FIG. 4.

FIG. 7 illustrates data sources of the federated learning model of FIG. 6.

FIG. 8 illustrates a process of the data processing system of FIG. 4.

FIG. 9 illustrates a process of the data processing system of FIG. 4.

FIG. 10 illustrates a process of the data processing system of FIG. 4.

FIG. 11 illustrates a process of the data processing system of FIG. 4.

FIG. 12 illustrates a process of the data processing system of FIG. 4.

FIG. 13 illustrates a process of the data processing system of FIG. 4.

FIG. 14 illustrates a process of the data processing system of FIG. 4.

FIG. 15 illustrates a process of the data processing system of FIG. 4.

FIG. 16 illustrates a process of the data processing system of FIG. 4.

FIG. 17 illustrates a process of the data processing system of FIG. 4.

FIG. 18 illustrates an example of a federated machine learning architecture according to another embodiment.

FIG. 19 illustrates a comparison between centralized, distributed, and federated learning according to another embodiment.

FIG. 20 illustrates an example of a semantic layer according to another embodiment.

FIG. 21 illustrates an example of a semantic layer according to another embodiment.

FIG. 22 illustrates an example of a semantic layer according to another embodiment.

### DETAILED DESCRIPTION

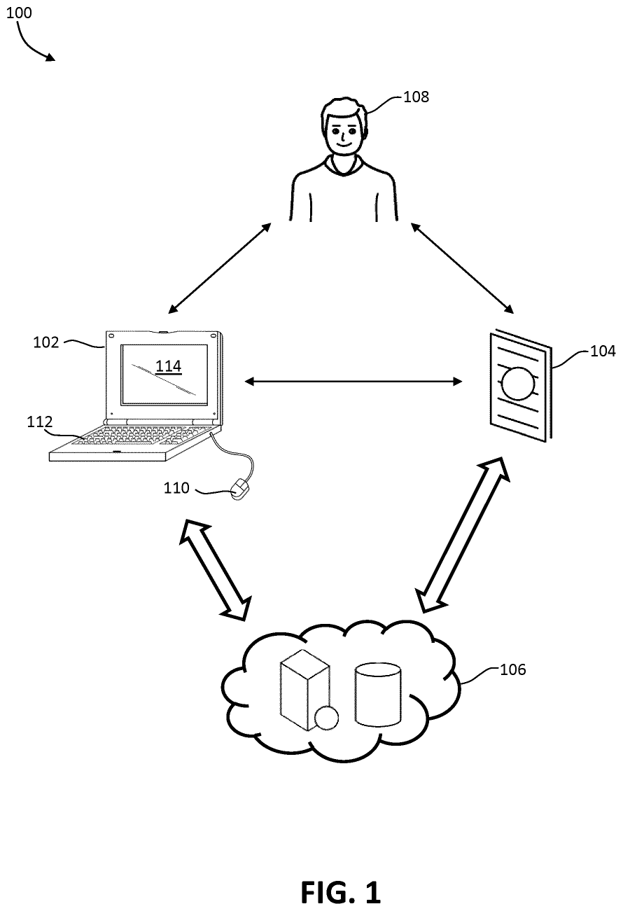

Referring to FIG. 1, a system for matching an individual to one or more opportunities according to one embodiment is shown generally at 100 and includes a user computing device 102, a measurement device 104, and a server computing device 106 operable to communicate with the user computing device 102 and the measurement device 104.

The user computing device 102 may be a personal computer, a laptop computer, a tablet computer, a smartphone, a smart watch, smart glasses, a mobile or a wearable activity or fitness tracker bearing electronic sensors (that may measure body movement) or multifunctional electronic circuit assemblies wearable or worn on the body, a haptic glove, or another computing device that includes one or more user input devices and one or more user output devices, and that is operable to provide an interactive user interface (such as an interactive user interface using a web page or an app, for example) on one or more output devices (such as a display screen or a video projector, for example) with a user 108 as described herein, for example. In the embodiment shown, the user computing device 102 is a laptop computer including a mouse 110, a keyboard 112, and a display screen 114.

The measurement device 104 is operable to measure one or more measurements of a body of the user 108, such as, for example, one or more haptic measurements, one or more biometric measurements, or one or more geolocation measurements. For example, the measurement device 104 may be operable to measure one or more of the user's heart rate, body temperature, respiratory rate, blood pressure, oxygen saturation, electrodermal activity, movement, posture, sleep pattern, nutrition intake, location, and environmental exposure, such as an environmental temperature, humidity, or air quality. The measurement device 104 may be wearable on the body of the user 108 when the measurement device 104 measures the one or more measurements. The measurement device 104 may be, for example, a haptic measurement device, a smartwatch, a fitness tracker, a heart rate monitor, a sleep monitor, a virtual reality headset, smart glasses, smart clothing, smart jewelry, smart shoes, or a smart hat.

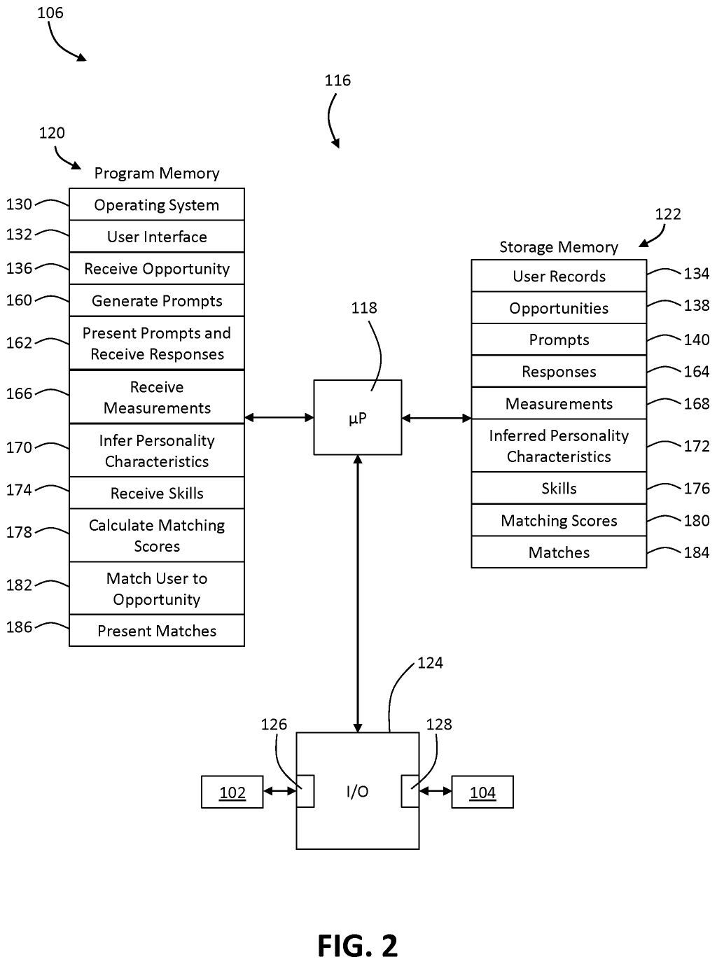

Referring now to FIG. 2, the server computing device 106 includes a processor circuit shown generally at 116. The processor circuit 116 includes a central processing unit (“CPU”) or microprocessor 118. The processor circuit 116 also includes a program memory 120, a storage memory 122, and an input/output (“I/O”) module 124 all in communication with the microprocessor 118. In general, the program memory 120 stores program codes that, when executed by the microprocessor 118, cause the processor circuit 116 to implement functions of the server computing device 106 such as those described herein, for example. Further, in general, the storage memory 122 includes stores for storing storage codes as described herein, for example. The storage memory 122 may store entries in tables of a relational database, for example. The program memory 120 and the storage memory 122 may be implemented in one or more of the same or different computer-readable storage media, which in various embodiments may include one or more of a read-only memory (“ROM”), random access memory (“RAM”), a hard disc drive (“HDD”), a solid-state drive (“SSD”), and other computer-readable and/or computer-writable storage media. The I/O module 124 may include various signal interfaces, analog-to-digital converters (“ADCs”), receivers, transmitters, and/or other circuitry to receive, produce, and transmit signals as described herein, for example. In the embodiment shown, the I/O module 124 includes a network interface 126 for transmitting signals to, and receiving signals from, the user computing device 102, and a network interface 128 for transmitting signals to, and receiving signals from, the measurement device 104. The network interfaces 126 and 128 may transmit signals to and receive signals from the user computing device 102 and the measurement device 104, respectively, using one or more networks such as the Internet, one or more wired networks, one or more wireless networks, or a combination of two or more thereof, for example.

The program memory 120 includes operating system program codes 130 of an operating system. The program memory 120 includes user interface program codes 132 that, when executed by the microprocessor 118, cause the processor circuit 116 to control an interactive user interface of the user computing device 102 by causing the network interface 126 to receive signals from the user computing device 102, and by causing the network interface 126 to transmit signals to the computing device 102. For example, if the user computing device 102 provides an interactive user interface using a web page, then the user interface program codes 132, when executed by the microprocessor 118, may cause the network interface 126 to transmit to the computing device 102 signals representing hypertext markup language (“HTML”) codes, JavaScript™ codes, or other codes that may control the interactive user interface, and may cause the network interface 126 to receive signals from the user computing device 102 representing user input in the interactive user interface.

In general, various users, including the user 108, may use such an interactive user interface to establish one or more user accounts, and storage codes representing details of such user accounts (such as usernames, passwords, and other user details) may be stored in a user records store 134 in the storage memory 122. The user 108 may be an individual seeking an opportunity, such as a job applicant or a student, for example, or an entity seeking to post an opportunity, such as an employer, a learning institution, or an educator, for example. Learning institutions or educators may include publicly funded learning institutions or educators, privately funded learning institutions or educators, or both. In general, learning institutions or educators as described herein may be independent of a provider of embodiments such as those described herein.

As used herein, an opportunity may include one or more learning opportunities and/or employment opportunities. A learning opportunity may include, for example, a learning institution or educator, a learning course, or a learning program including more than one learning course, and a learning course may be part or all of a formal course or may include one or more electronic media resources (or stories) such as videos, podcasts, written articles, or webinars. One or more hashtags may be associated with one or more of such electronic media resources. In some embodiments, a learning opportunity may be a remote learning opportunity. An employment opportunity may include, for example, a job posting. In some embodiments, an opportunity may include an employment opportunity and one or more relevant learning opportunities that provide knowledge associated with the employment opportunity. For example, the one or more relevant learning opportunities may provide education, training, and/or credentials that may be required for the employment opportunity. That is, the one or more relevant learning opportunities may provide knowledge to meet skill demands or bridge skill gaps for the employment opportunity.

The program memory 120 may include receive opportunity program codes 136 that, when executed by the microprocessor 118, cause the processor circuit 116 to control an interactive user interface of a user computing device (such as the user computing device 102) that allows a user such as an employer, a learning institution, or an educator to post an opportunity by providing various details of the opportunity. For employment opportunities, such details may include a job description, a location of the job (if any), and an identification of one or more skills and preferred personality characteristics that are desirable for job applicants for the employment opportunity. For learning opportunities, when a learning opportunity is a learning institution or educator, the details may include a description of the learning institution or educator and an identification of one or more skills and/or preferred personality characteristics that identify a likely benefit from the learning institution or educator. When a learning opportunity is a learning course, or a learning program including more than one learning course, the details may include a description of the learning course or learning program and an identification of one or more skills and/or preferred personality characteristics that identify a likely benefit from the learning course or learning program. Representations of such details of opportunities may be stored in an opportunities store 138 in the storage memory 122.

The storage memory 122 may also include a prompts store 140 storing representations of prompts to be presented to users such as job applicants or students, and responses obtained from those users in response to the prompts. In general, each prompt may be a question that can be posed to a job applicant or student and that may facilitate determining the suitability of the job applicant or student for one or more opportunities (such as an opportunity having details stored in the opportunities store 138), for example.

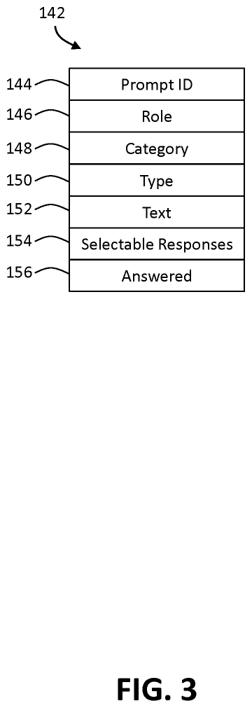

The prompts store 140 may include a table that may store any number of instances of a prompts table entry shown generally at 142 in FIG. 3. In general, the prompts table entry 142 may include various fields as described below. Each instance of the prompts table entry 142 may be associated with a respective prompt and can store, in such fields, particular values associated with the respective prompt. The prompts table entry 142 includes a prompt identifier field 144, which stores an integer that may be assigned by database management system (“DBMS”) codes to identify an instance of the prompts table entry 142 uniquely in the prompts store 140.

The prompts table entry 142 also includes a role field 146 that may store a representation of a role or type of user, such as a representation of job applicants, a representation of students, a representation of employers, or a representation of learning institutions or educators, for example. As a result, data in the role field 146 of an instance of the prompts table entry 142 may indicate that a prompt associated with an instance of the prompts table entry 142 is intended to be asked of a particular type of user, such as a job applicant, a student, an employer, a learning institution, or an educator, for example.

The prompts table entry 142 also includes a category field 148 that may store a representation of a category of a prompt associated with an instance of the prompts and selectable responses table entry 142. For example, some prompts may be associated with open-ended responses and such prompts may be in a category of prompts that are associated with open-ended responses. Examples of prompts that are associated with open-ended responses include “Tell us about your volunteer activities.” and “What type of outdoor activities do you enjoy?” Such prompts may allow a respondent to fill in a blank space. Other prompts may be associated with selectable or fixed responses and such prompts may be in a category of prompts that are associated with selectable or fixed responses. For example, a prompt “Were you on a team or a solo volunteer?” may be associated with two selectable responses, namely “Team Volunteer” and “Solo Volunteer”.

The prompts table entry 142 also includes a type field 150 that may store a representation of a type of a prompt associated with an instance of the prompts and selectable responses table entry 142. A type may be positive, such as “Pick the industry sector you are more interested in.” or “What job benefits would you like?”, or negative, such as “Which skill do you think is the most useless?” or “Which occupation would be a terrible fit for you?”.

The prompts table entry 142 also includes a text field 152 that may store a representation of text of a prompt associated with an instance of the prompts table entry 142, such as “Tell us about your volunteer activities.” or “Were you on a team or a solo volunteer?”.

The prompts table entry 142 also includes a selectable responses field 154 that may store representations of selectable responses for prompts that are associated with selectable or fixed responses. For example, for the prompt “Were you on a team or a solo volunteer?”, the selectable responses field 154 may store the selectable responses “Team Volunteer” and “Solo Volunteer”.

The prompts table entry 142 also includes an answered field 156 that may store representations of when a prompt associated with an instance of the prompts table entry 142 has been answered.

Referring back to FIG. 2, in some embodiments, the program memory 120 may include generate prompts program codes 160 that, when executed by the microprocessor 118, cause the processor circuit 116 to generate prompts to be presented to users based on, for example, details included in one or more opportunities of the opportunities store 138, and to store the generated prompts in the prompts store 140. For example, the generate prompts program codes 160, when executed by the microprocessor 118, may cause the processor circuit 116 to dynamically generate prompts using generative AI or AI-generated media, all within the system 100 (i.e., without leaving the system 100). Such generative AI may include one or more of a large language model (such as ChatGPT, Google Bard, Jasper, Persado, or Baidu Ernie), a text-to-image model (such as Dall-E, Stable Diffusion, Midjourney, or OpenArt), a text-to-speech model or text-to-video model (such as Make-A-Video, Runway, Fliki, or Resemble AI), and an AI-based code completion tool (such as Tabnine, GitHub Copilot, Replit, or mutable.ai).

Still referring to FIG. 2, the program memory 120 may include present prompts and receive responses program codes 162 that, when executed by the microprocessor 118, cause the processor circuit 116 to control an interactive user interface of a user computing device (such as the user computing device 102) to present prompts to a job applicant or student user, receive responses to the prompts from the job applicant or student user, and store the responses in a responses store 164 in the storage memory 122.

For example, where a prompt associated with open-ended responses, the present prompts and receive responses program codes 162, when executed by the microprocessor 118, may cause the processor circuit 116 to send out at least one signal from the network interface 126 to a user computing device, such as the user computing device 102, to control an interactive user interface of the user computing device to cause a display screen, a video projector, or another output device of the user computing device, such as the display screen 114 of the user computing device 102, to present text of the prompt (which may be retrieved from the text field 152 of an instance of the prompts table entry 142 associated with the prompt) and to present a space for the user to enter a response. Again, a selectable icon may also allow the user to skip the prompt, or the user may otherwise be presented with an option of skipping the prompt. Using one or more input devices of the user computing device, such as the mouse 110 and keyboard 112 of the user computing device 102, the user may enter a response or skip the prompt, and at least one signal representing the user response (whether indicating a response or skipping the prompt) may be transmitted from the user computing device to the network interface 126.

Similarly, where a prompt is associated with selectable or fixed responses, the present prompts and receive responses program codes 162, when executed by the microprocessor 118, may cause the processor circuit 116 to send out at least one signal from the network interface 126 to a user computing device, such as the user computing device 102, to control an interactive user interface of the user computing device to cause a display screen, a video projector, or another output device of the user computing device, such as the display screen 114 of the user computing device 102, to present text of the prompt (which may be retrieved from the text field 152 of an instance of the prompts table entry 142 associated with the prompt) and to present the selectable responses (which may be retrieved from the selectable responses field 154 of the instance of the prompts table entry 142 associated with the prompt) in respective selectable icons, or otherwise selectable. A selectable icon may also allow the user to skip the prompt, or the user may otherwise be presented with an option of skipping the prompt. Using one or more input devices of the user computing device, such as the mouse 110 and keyboard 112 of the user computing device 102, the user may select one of the selectable responses or skip the prompt, and at least one signal representing the user response (whether indicating selection of one of the selectable responses or skipping the prompt) may be transmitted from the user computing device to the network interface 126.

Therefore, the processor circuit 116 may send out at least one outgoing output signal including at least prompt text as described above to an output device, such as the display screen 114 of the user computing device 102, and may receive at least one incoming input signal representing responses to respective prompts as described above from an input device, such as the mouse 110 and keyboard 112 of the user computing device 102.

The program memory 120 may further include receive measurements program codes 166 that, when executed by the microprocessor 118, cause the processor circuit 116 to receive measurements of a body of a job applicant or student user from a measurement device, such as the measurement device 104, and store the measurements in a measurements store 168 in the storage memory 122. For example, the receive measurements program codes 166, when executed by the microprocessor 118, may cause the processor circuit 116 to send out at least one signal from the network interface 128 to a measurement device, such as the measurement device 104, to cause the measurement device to measure one or more measurements of the body of the job applicant or student user. As described above, such measurements may include the user's heart rate, body temperature, respiratory rate, blood pressure, oxygen saturation, electrodermal activity, movement, posture, sleep pattern, nutrition intake, location, and environmental exposure, such as an environmental temperature, humidity, or air quality. Once a measurement has been made, at least one signal representing the measurement may be transmitted from the measurement device to the network interface 128.

The program memory 120 may further include infer personality characteristics codes 170 that, when executed by the microprocessor 118, cause the processor circuit 116 to obtain one or more responses corresponding to a job applicant or student user from the responses store 164 and one or more measurements of a body of the job applicant or student user from the measurements store 168, and to infer one or more personality characteristics of that job applicant or student user from the one or more responses and the one or more measurements. Representations of the inferred personality characteristics may be stored in an inferred personality characteristics store 172 in the storage memory 122. In general, a personality characteristic may be one or more of a personality type, such as a Holland personality type (i.e., a Holland Code), a personality trait, and a personality state. Inferring one or more personality characteristics of a job applicant or student user may involve, for example, for each personality characteristic of a defined set of personality characteristics, such as the Holland Codes, calculating a respective probability that the user belongs to or has that personality characteristic, and ranking the personality characteristics of the defined set according to their respective probabilities. The ranking may then be used to categorize the user as belonging to or having one or more of the personality characteristics of the defined set (e.g., the highest ranked personality characteristic of the set).

In general, a probability that the job applicant or student user belongs to or has particular personality characteristic may be determined from the responses of the user by associating pre-defined responses or types of responses with specific personality characteristics. For example, some responses may indicate that the user has one or more interests or sentiments, and some of those interests or sentiments may be associated with specific personality characteristics. More specific examples are provided in Table 1.

TABLE 1
Examples of inferring personality characteristics from interests or sentiments

| | | Sentiment/Interests/CODE | | | | | |
|---|---|---|---|---|---|---|---|
| | | Builder | Analyzer | Creator | Organizer | Dealer | Socializer |
| Question Set | | B | A | C | O | D | S |
| 1) | Do you love being | Camping, | Programming | Create | Organize | Road sports, | Create |
| | outdoors in nature? | Hiking, | Activities | Food | Activities | Cycling, | Entertainment |
| | Share the type of | Fishing, | | | | Water | |
| | activities you do. For | Water | | | | Sports, | |
| | example, camping, | Sports | | | | Running | |
| | cycling hiking, water | | | | | Marathons | |
| | sports, road sports, | | | | | | |
| | running marathons. Or | | | | | | |
| | would you rather pitch in | | | | | | |
| | to create the food, or | | | | | | |
| | create the entertainment, | | | | | | |
| | or organize the activities | | | | | | |
| | or to program activities? | | | | | | |
| | Share all the items you | | | | | | |
| | like to do or can do. | | | | | | |
| 2) | Do you feel relaxed, | Attend | Attend Solo, | Write or | Attend | Attend in | Attend in |
| | exited or good when you | concerts, | Virtually | Compose | virtually or | person, | person with |
| | attend concerts or | theatre, | | own, | in-person | hockey | Group |
| | theatre? Or would you | drama | | attend in | with Group | games | |
| | rather write or compose | | | person | | | |
| | your own? Do like to | | | | | | |
| | attend solo or with a | | | | | | |
| | group? Opera, Rock or | | | | | | |
| | Classical? Would you | | | | | | |
| | attend in-person or | | | | | | |
| | virtually? | | | | | | |
| 3) | Tell us about your | Building | Solo | Solo or | Team | Solo | Team or Solo |
| | volunteer activities, | shelters, | Volunteer, | Group | Volunteer | Volunteer, | Volunteer, |
| | clubs? Were you on a | Sports | bookkeeping | Volunteer, | | Political, | Club, Team, |
| | team or a solo volunteer? | events | | Cooking | | Politics, | education, |
| | What type of activity? | | | Meals | | Debate, | Healthcare |
| | Building shelters for | | | | | Sports | |
| | people, doing | | | | | Events | |
| | bookkeeping, cooking | | | | | | |
| | meals, health, education | | | | | | |
| | or sports or political | | | | | | |
| | event? List all that you do | | | | | | |
| | or have done or would | | | | | | |
| | like to do. | | | | | | |

For example, considering question set 1 in Table 1, when presented with a prompt such as “Do you love being outdoors in nature? Share the type of activities you do. For example, camping, cycling hiking, water sports, road sports, running marathons. Or would you rather pitch in to create the food, or create the entertainment, or organize the activities or to program activities? Share all the items you like to do or can do.”, a user response of “Camping, Hiking, Fishing, Water Sports” may be associated with a “Builder” personality characteristic, a user response of “Programming Activities” may be associated with an “Analyzer” personality characteristic, a user response of “Create Food” may be associated with an “Creator” personality characteristic, a user response of “Organize Activities” may be associated with an “Organizer” personality characteristic, a user response of “Road sports, Cycling, Water Sports, Running Marathons” may be associated with an “Dealer” personality characteristic, and a user response of “Create Entertainment” may be associated with an “Socializer” personality characteristic.

As illustrated in in Table 1, within a defined set of personality characteristics, such as the set (“Builder”, “Analyzer”, “Creator”, “Organizer”, “Dealer”, “Socializer”), each personality characteristic may be assigned a personality characteristic code. For example, “Builder” may be assigned code B, “Analyzer” may be assigned code A, “Creator” may be assigned code C, “Organizer” may be assigned code O, “Dealer” may be assigned code D, and “Socializer” may be assigned code S.

User responses may point to multiple personality characteristics. For example, a user response to question set 1 from Table 1 indicating that the user likes to be outdoors and hike may be associated with both the “Builder” and “Creator” personality characteristics (code BC). Similarly, a user response to question set 2 from Table 1 indicating that the user feels most relaxed attending in-person at soft rock concerts with a group of friends may be associated with the “Builder”, “Socializer”, and “Dealer” personality characteristics (code BSD); and a user response to question set 3 from Table 1 indicating that the user likes to volunteer as a solo, and has done bookkeeping for a not-for-profit in sports may be associated with the “Analyzer” and “Socializer” personality characteristics (code AS).

Further, multiple responses (and thus, multiple prompts) may be required to infer the one or more personality characteristics. For example, in some embodiments, a job applicant or student user may be presented with five or more prompts, and may be required to provide five or more corresponding responses.

The measurements of the body of the job applicant or student user may be used to corroborate or verify one or more of the user's responses when inferring that user's personality characteristics. For example, measurements of the user's heart rate, respiratory rate, blood pressure, electrodermal activity, and/or posture may be used to confirm the user's expressed interest in activity such as a sporting event or hobby, and measurements of the user's geolocation may be used to confirm the user's attendance at events that the user indicated that they had attended. The measurements of the body of the job applicant or student user may also be used to determine weightings, such as normalized weightings, to be attached to one or more of the user's responses or to one or more personality characteristics of a defined set of personality characteristics. For example, an increasing adjustment weighting may be attached to a positive response regarding an activity that measurements of the user's body (e.g., heart rate, respiratory rate, blood pressure, electrodermal activity, and/or posture) indicate that the user enjoyed. Such weightings can be used, for example, to weight the responses and/or personality characteristics to which they are attached when calculating probabilities that the user belongs to or has particular personality characteristics, as described above.

In some embodiments, the program memory 120 may further include receive skills program codes 174 that, when executed by the microprocessor 118, cause the processor circuit 116 to control an interactive user interface of a user computing device (such as the user computing device 102) that allows a user such as a job applicant or student to submit information regarding their skills that may be relevant to one or more opportunities. For example, the skills information may include one or more of credentials obtained, courses completed, and work experience. Representations of such skills information may be stored in a skills store 176 in the storage memory 122.

The program memory 120 may further include calculate matching scores codes 178 that, when executed by the microprocessor 118, cause the processor circuit 116 to calculate one or more matching scores generally representing a degree of matching between a user such as a job applicant or student and one or more opportunities. Representations of the matching scores may be stored in a matching scores store 180 in the storage memory 122. A matching score for a pair of a user and an opportunity may represent, for example, a pairing of one or more inferred personality characteristics of the user with one or more preferred personality characteristics of the opportunity. Therefore, to determine a matching score for a pair of a user and an opportunity, the calculate matching scores codes 178, when executed by the microprocessor 118, may cause the processor circuit 116 to obtain one or more preferred personality characteristics aligned or associated with the opportunity from the opportunities store 138 and one or more inferred personality characteristics aligned or associated with the user from the inferred personality characteristics store 172, and compare the one or more inferred personality characteristics of the user to the one or more preferred personality characteristics of the opportunity to calculate the matching score. Where an opportunity has more than one aligned or associated preferred personality characteristic, the aligned or associated preferred personality characteristics may be ranked in order of preference, with the order of preference included in the details associated with the opportunity and stored in the opportunities store 138. In such cases, the one or more inferred personality characteristics of the user may be compared to each of the aligned or associated preferred personality characteristics of the opportunity, while accounting for the ranking. For example, a matching score for a pairing of a user “j” and an opportunity “k” may be calculated as:

$$Matching Score ( {user}_{j} , {opportunity}_{k} ) = {\sum }_{i = 1}^{n} [ P ( {type}_{i} , {user}_{j} ) \times R ( {type}_{i} , {opportunity}_{k} ) ]$$

where P (typeᵢ, userⱼ) is a probability that the user “j” belongs to or has a personality type “i”, R (typeᵢ, opportunityₖ) is a ranking or preference of the opportunity “k” for the personality type “i”, and n is the number of personality types considered.

As a more specific example of matching a user with an opportunity, and referring back to Table 1 and the associated example personality characteristics, consider the opportunity having a ranked personality preference of BCS (i.e., “Builder”, then “Creator”, then “Socializer”). If the user is presented is with the prompts of question set 1 from Table 1 and responds indicating that they like to be outdoors and hike, then, as described above, it may be inferred that the user has both the “Builder” and “Creator” personality characteristics (code BC). As such, the user's inferred personality characteristics would match two of three of the opportunity's preferred personality characteristics.

Similarly, consider an opportunity having a ranked personality preference of BDS. In that case, if the user is presented with the prompts of question set 2 from Table 1 and responds indicating that they feel most relaxed attending in-person at soft rock concerts with a group of friends, then, as described above, it may be inferred that the user has the “Builder”, “Dealer”, and “Socializer” personality characteristics (code BDS). As such, the user's inferred personality characteristics would match all three of the opportunity's preferred personality characteristics.

Similarly, consider an opportunity having a ranked personality preference of AOS. In that case, if the user is presented with the prompts of question set 3 from Table 1 and responds indicating that they like to volunteer as a solo, and have done bookkeeping for a not-for-profit in sports, then, as described above, it may be inferred that the user has the “Analyzer” and “Socializer” personality characteristics (code AS). As such, the user's inferred personality characteristics would match two of three of the opportunity's preferred personality characteristics.

In some embodiments, a matching score for a pair of a user and an opportunity may represent a pairing of one or more inferred personality characteristics of the user and one or more skills of the user with one or more preferred personality characteristics aligned or associated with the opportunity and one or more skills required for the opportunity. In such embodiments, to determine a matching score for a pair of a user and an opportunity, the calculate matching scores codes 178, when executed by the microprocessor 118, may cause the processor circuit 116 to obtain one or more preferred personality characteristics aligned or associated with the opportunity and one or more skills required for the opportunity from the opportunities store 138, one or more inferred personality characteristics aligned or associated with the user from the inferred personality characteristics store 172, and one or more skills of the user from the skills store 176, and may cause the processor circuit 116 to compare the one or more inferred personality characteristics and the one or more skills of the user to the one or more preferred personality characteristics of the opportunity and the one or more skills required for the opportunity to calculate the matching score.

Once calculated, matching scores may be used to match users to opportunities. Accordingly, the program memory 120 may further include match user to opportunity program codes 182 that, when executed by the microprocessor 118, cause the processor circuit 116 to match one or more job applicant or student users to one or more opportunities based on respective matching scores representing pairings between the users and the opportunities. Representations of such matches may be stored in a matches store 184 in the storage memory 122. For example, given a set of job applicant or student users, a set of opportunities, and a set of respective matching scores representing each possible pairing of user and opportunity from the user set and the opportunity set, as calculated using the calculate matching scores program codes 178 as described above, the match user to opportunity program codes 182, when executed by the microprocessor 118, may cause the processor circuit 116 to identify matches between users and opportunities based on the matching scores. A match may be indicated, for example, by a highest matching score value. Thus, where a given job applicant or student user is considered for multiple opportunities, each of the opportunities having a corresponding matching score with the user, the user may be matched to whichever opportunity corresponds to the highest matching score value. Similarly, where multiple job applicant or student users are considered for a single opportunity, ach of the users having a corresponding matching score with the opportunity, whichever user corresponds to the highest matching score value may be matched to the opportunity.

In some embodiments, the program memory 120 may further include present matches program codes 186 that, when executed by the microprocessor 118, cause the processor circuit 116 to control an interactive user interface of a user computing device (such as the user computing device 102) to present matches between job applicant or student users and opportunities to a user. For example, for a job applicant or student user, the present matches program codes 182, when executed by the microprocessor 118, may cause the processor circuit 116 to obtain the user's own matches to opportunities from the matches store 184 and present those opportunities to the user. In some embodiments, postings and details of an opportunity may be withheld from a job applicant or student user unless that user is matched to that opportunity. That is, the opportunity may only be presented to the user once a match has been made.

Of course, the embodiments described above are examples only, and alternative embodiments may vary. For example, in some embodiments, instead of the single user computing device 102 and the single measurement device 104, the system 100 may include multiple user computing devices, multiple measurement devices, or both multiple user computing devices and multiple measurement devices. Further, in some alternative embodiments, the measurement device 104 may communicate directly with the user computing device 102. Still other alternative embodiments may infer personality characteristics of users based only on user responses, rather than on user responses and body measurements.

In some alternative embodiments, a matching score for a pair of a user and an opportunity may represent a degree of matching between a user such as an educator and one or more employment opportunities that require skills that the educator may provide. Thus, an educator user can get real-time data on employer skill demands, which may help to reduce a lag time (of normally 6 months to a year) for educators to update course content and delivery mode preferences based on the employer information.

In some alternative embodiments, employer users may also search for both candidates that match employer skill requirements for employment opportunities posted by the employer, as well as courses that provide one or more required skills for each employment opportunity. This functionality may be important as the courses may provide needed upskilling, reskilling, or training to meet the skill requirements for the employment opportunities. This functionality may also helps to verify or prequalify/prescreen job applicant users/candidates in the system 100 for the employment opportunity.

### Additional Examples

The following embodiments are provided as non-limiting examples.

At least one embodiment may include a computer-implemented method in a data processing system that suggests career candidates, career skills, career occupations, career courses, Stories (text, voice, or video, for example), or events for a target user based on stored data in the data processing system related to the target user seeking careers, skills, courses, events, or career candidates simultaneously across industry sectors. The data processing system may suggest careers, skills, courses, career candidates, or events based on the target user's affinity for, connections with, or interactions with other users, stories, or objects (such as Haptic human-machine interfaces (HMI) including Wearable Internet of Things (WIoT) biometric feedback and/or Immersive Reality events such as augmented reality (A/R), virtual reality (V/R), mixed reality (M/R), or extended reality (X/R) experience reactions) in the data processing system connected to or otherwise associated with the career postings, course postings, stories, or events. For example, a course posting is suggested to a target user if users viewed a career posting, or viewed a story or an event. As another example, an event organized by a particular entity may be suggested to the target user because of interactions between the target user and other content provided by the entity in the data processing system. Suggested events may be presented to the target user via a client device, allowing the target user to easily join a suggested event.

At least one embodiment may include a computer-implemented method in a multi-mode interoperable data processing system for workforce advancement across industry sectors in real time.

At least one embodiment may include a computer-implemented method comprising: accessing user profile information in a data processing system containing information about types of target users, wherein the information comprises information relating to the target job candidate user's profile to seek career opportunities and to advance skills, or relating to target employers to recruit job candidates or to advance workforce skills, or relating to target educators to align courses to skills—to deliver results to each user target type simultaneously and all in one go; retrieving data stored in the data processing system related to a plurality of career advancement queries, each query associated with at least one of a time, user profiles, a job posting, course posting, location, event, or story and a description; selecting one or more candidate queries, based on a connection between the target user and the query, using the user profile information relating to the target user and the data related to the plurality of queries; generating, by a processor, a candidate query relevance score for each candidate query based on the user profile information including the target user's history of attending events, or viewing stories (articles or videos) and queries on courses, jobs, articles & videos and the data associated with the candidate query, the candidate query relevance score associated with each candidate query, comprising an estimate of a probability that the target user will attend the candidate event, or course or apply for the job posting, responsive to a suggestion; selecting an event, or course or job posting from the candidate queries based on the candidate query relevance scores; and suggesting the selected event, course, job posting, article, or video to the target user by presenting it to the user in one or more user interfaces of the multi-mode model data processing system.

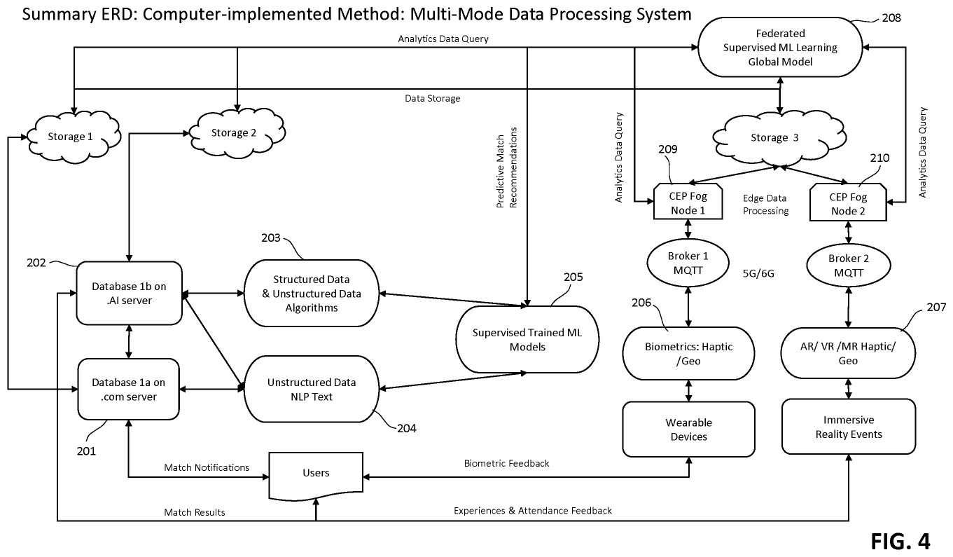

In some embodiments, the computer-implemented method of use optimizes the latency, interoperability and access to a Federated ML Learning cloud architecture and systems (See FIG. 4) for workforce development: comprising 1) a data processing system (I. computer-implemented method noted above) and 2) the biofeedback data from body worn computer devices and sensors (WIOT) using SND (software defined networking) innovations to support 5G and beyond mobile networks.

Such embodiments may optimize operating performance by reducing latency.

Such embodiments may optimize Federated Model Multi-Cloud infrastructure interoperability for the integration of WIOT (Wearable Internet of Things) data sharing and data processing

Such embodiments may optimize software as a service (SaaS), infrastructure as a service (IAAS) & platform as a service (PAAS) integration to reduce operating costs and improve affordable access for workforce advancement

FIG. 4 illustrates one embodiment. In FIG. 4, Data Processor 201 for FutureCite.com is the site with the stories (articles, videos, & podcasts) platform, events, and networking platform which includes connecting mentors & proteges and forum (plugin), and primary signup registration site for all users and as well the paywall (user information).

Data Processor 202 for FutureCite.ai is the site with user detailed profiles of jobseekers, employers/recruiters, and educators which includes structured data 203 (current matching algorithms) and unstructured data 204 (existing long form questions). The unstructured data using natural-language processing (NLP) is addressed in the following additional subject matter.

The embodiment of FIG. 4 may combining all three fields of use i.e. jobseekers, educators and employers-matching their needs simultaneously and all in one go to advance workforce development. A computer-implemented data processing method may look at MULTI-MODE and MULTI-LEVEL data by adding the machine learning (ML) computer models for Natural Language Processing (NLP) in Level 1, and the ML models for the addition of wearables 206 in Level 2 (biometrics & haptic feedback) and the ML models for the addition of experiences through (AR/VR/MR/XR) immersive reality events 207 in Level 3.

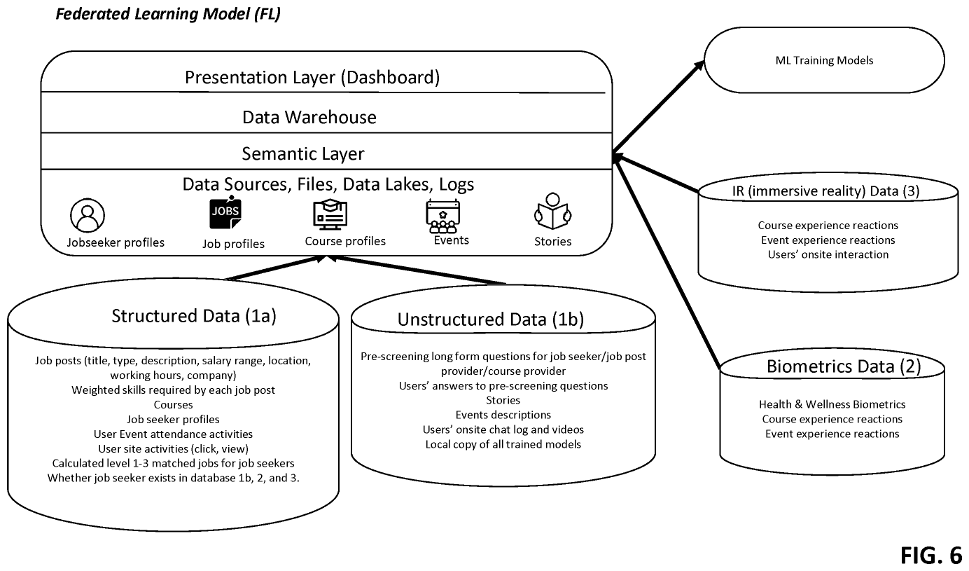

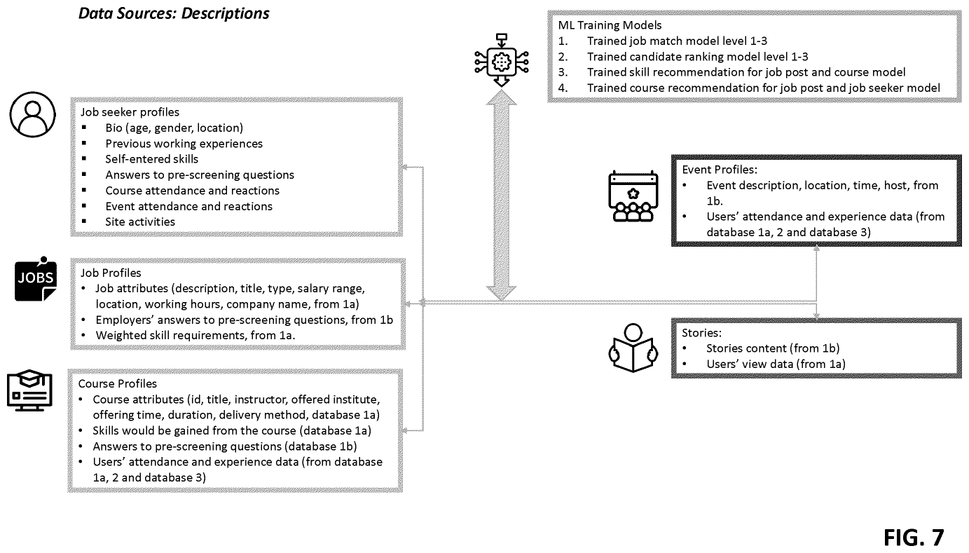

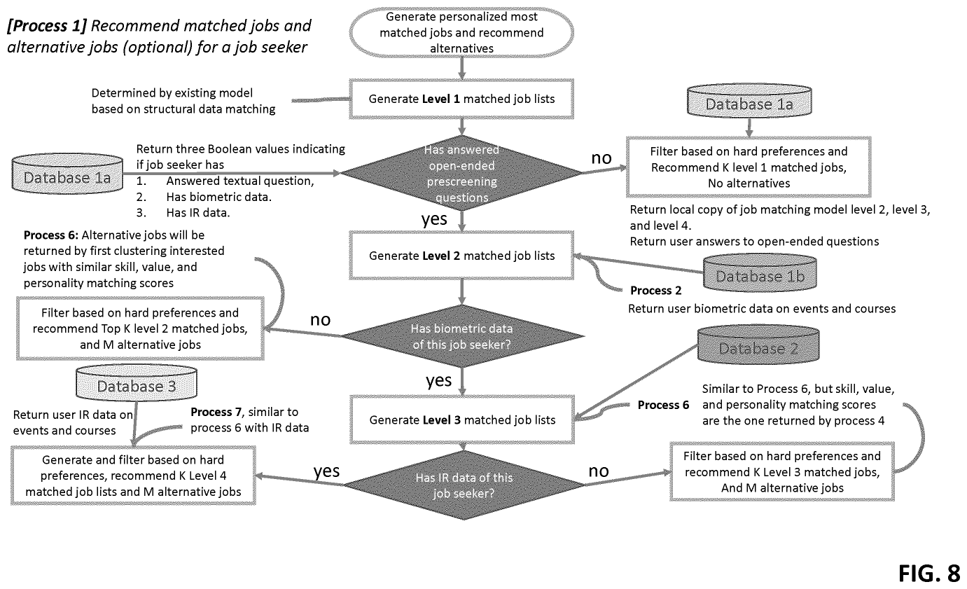

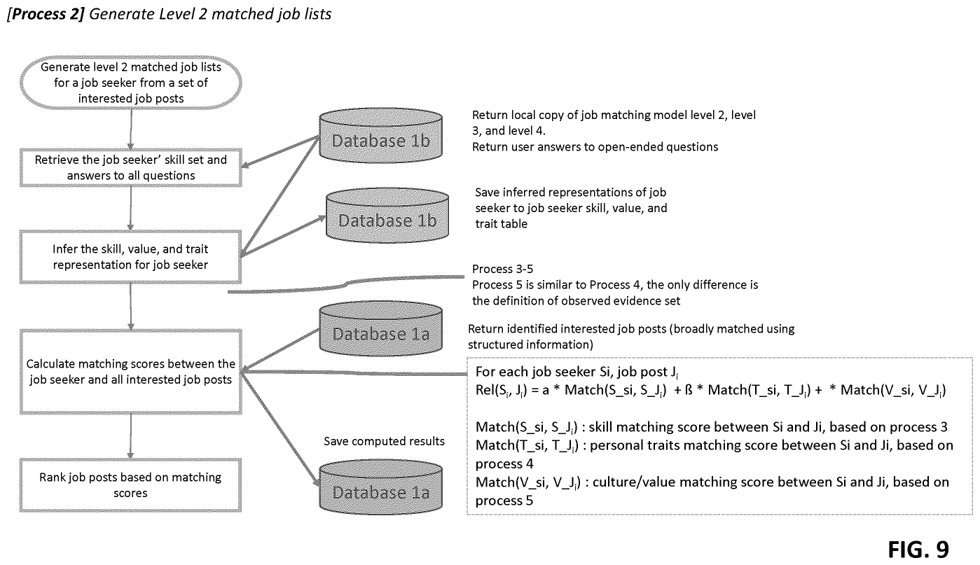

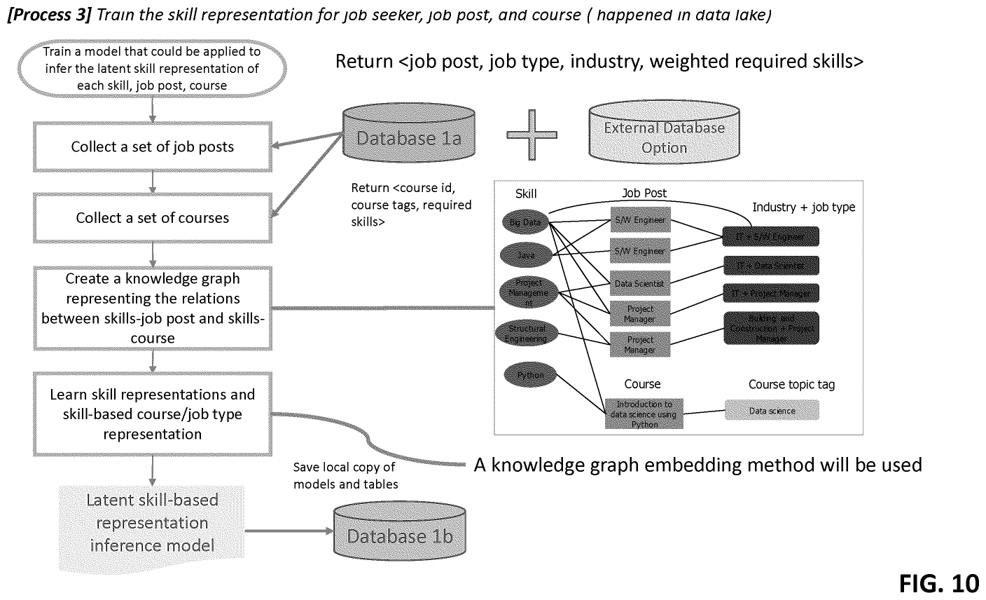

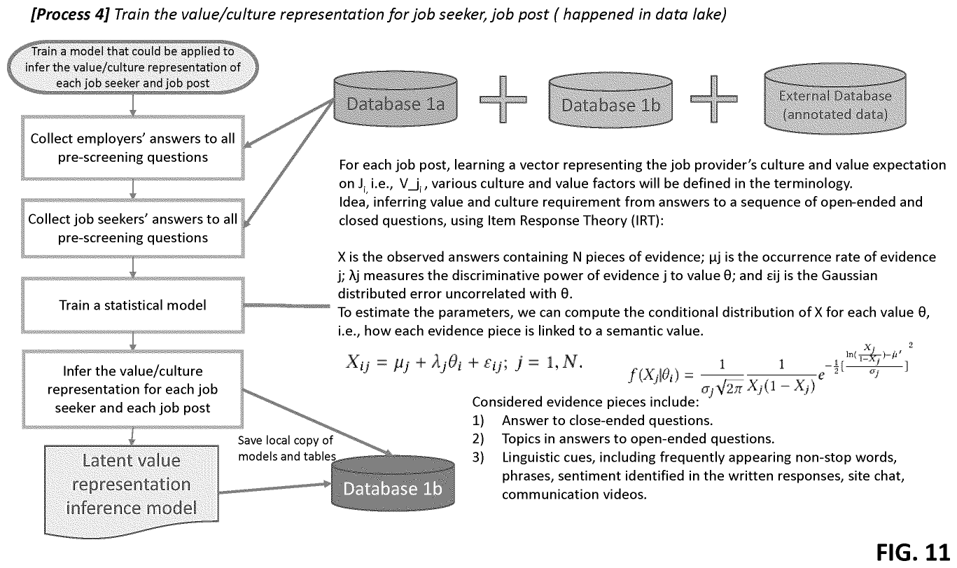

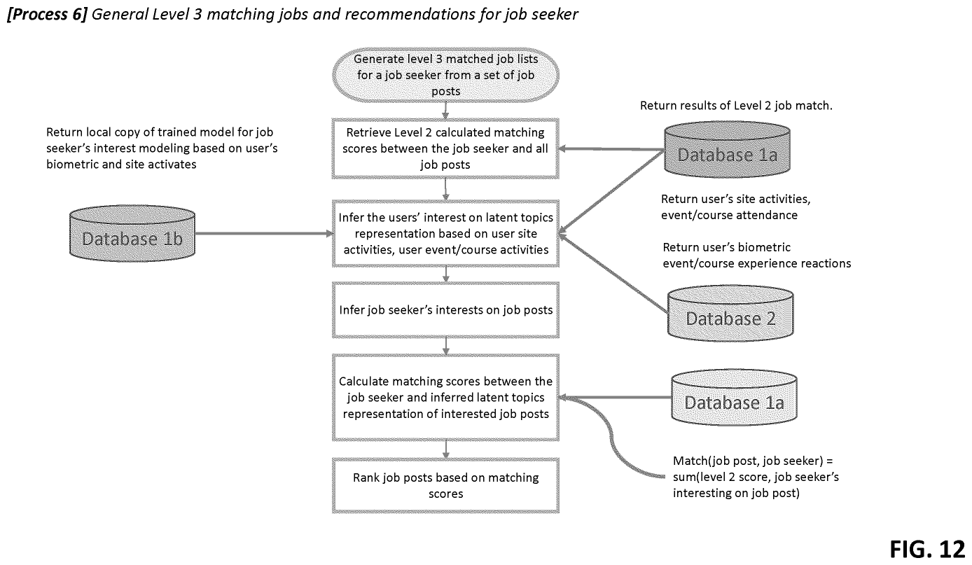

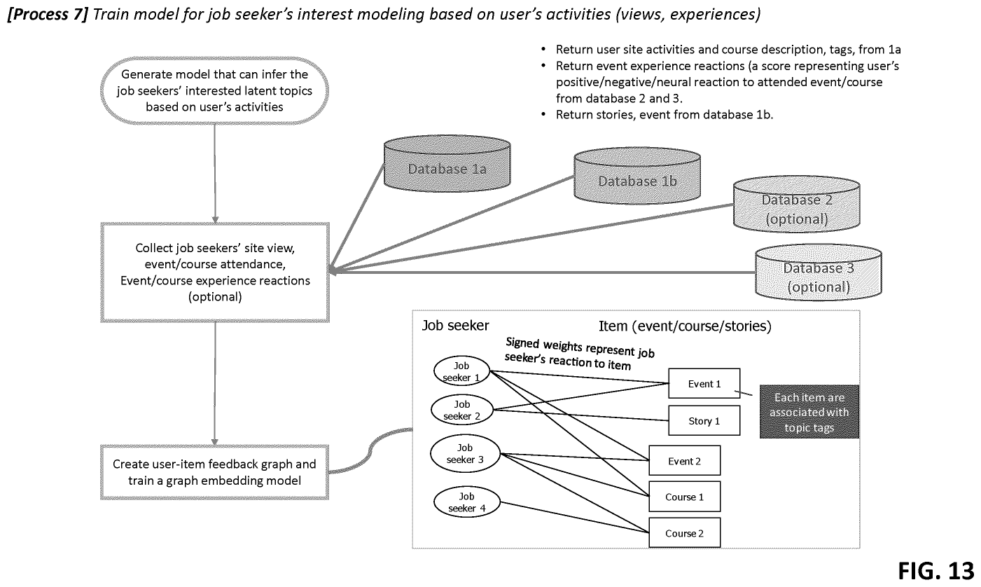

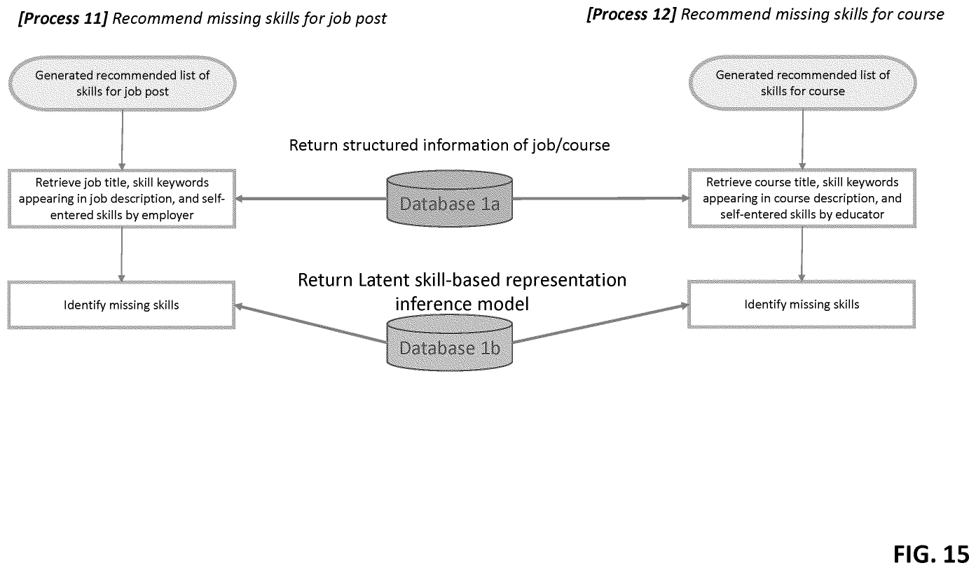

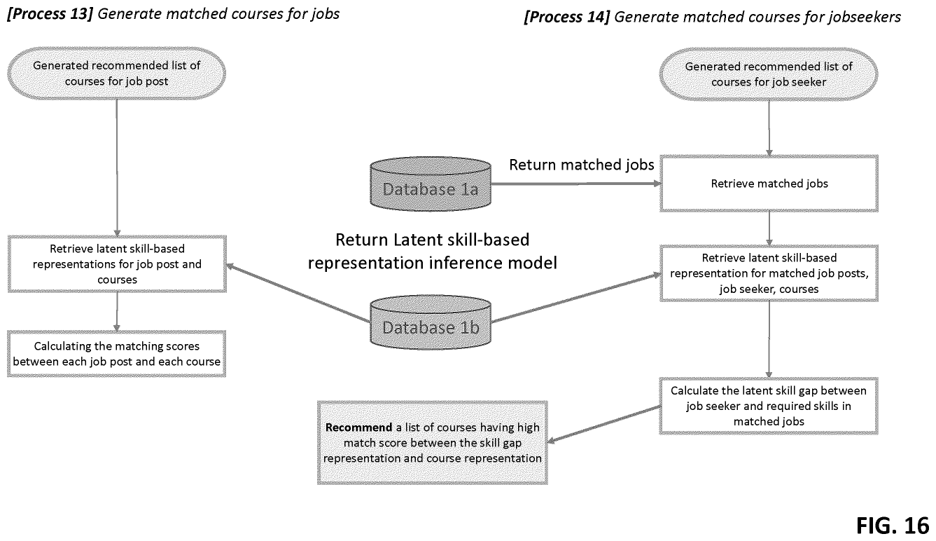

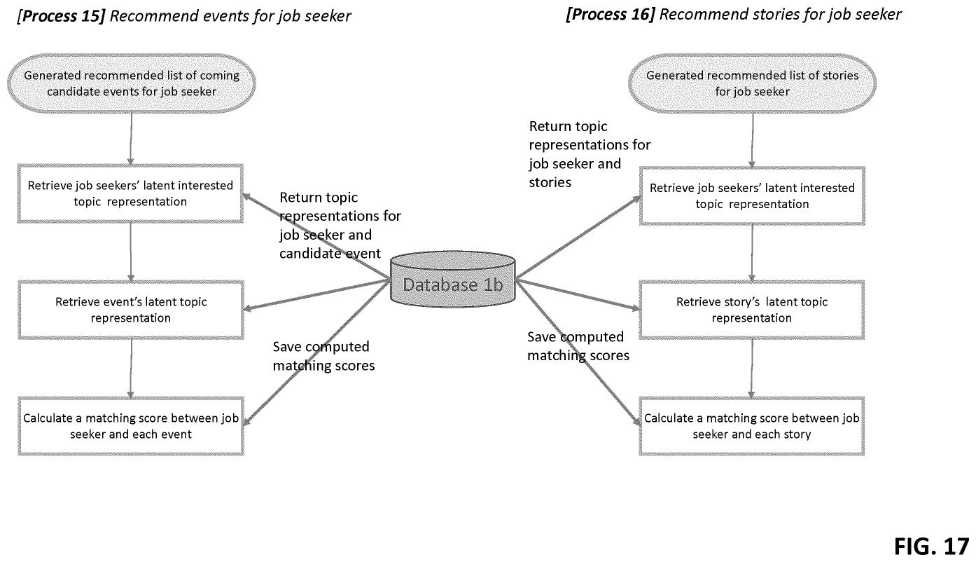

The assessments & matching processes (as described in PCT/CA2021/051207) with the additional queries, data, and analytics extracted from the artificial intelligence (AI) (ML models) 208 from PCT/CA2021/051207 to the addition of data information and results from Level 1 to Level 3 described in the following (and as illustrated FIGS. 6 to 17) may provide the users with enhanced prescreening and assessment matching to provide predictive recommendations which may be additional options to users that may not otherwise be presented to them through traditional human resources (HR) prescreening methodologies, and/or through traditional education pathway methodologies.

Most current workforce development programs (which may be SaaS) are designed for intracompany employee advancement, but not for advancement outside of the employee's current company to a different and unrelated industry sector.

At least one embodiment may include a self-navigating one stop, all in one go process that may reduce time and costs for employers, jobseekers and educators. In some embodiments: a Jobseeker may transition from one occupation to another, or from one company to another in a different sector/job to move to another industry sector, and find courses (whether in-person, remote, or hybrid learning options) to upskill, reskill and train—to match their schedule, costs, and mode of delivery; an Employer may better identify job candidates with transferable skills from one occupation to another, and from one industry sector to another-reducing costs to hire and retain staff (reduce churn in organizations); an Educator may identify skills needs or discover new skills needs in real time—to improve course content, context, and mode of delivery and skills deliverables to better align with needs of Employers.

### Overview of Some Embodiments

First, some embodiments in workforce development may enable all the three user types (Jobseekers, Employers, Educators) to self-navigate from one industry sector to another, with a Multi Level Model (MLM) assessment that may provide prescreening and matching recommendations for candidates, careers/jobs, and courses-simultaneously across all industry sectors and all in real-time without having to leave our system. An AI model may generate three additional levels of assessment matches of Level 1, Level 2 and Level 3 to a current innovation Level 0 (Database 1a as shown in FIG. 4) to provide additional recommendations to the users (Jobseekers, Employers, Educators). In some embodiments, Level 1 best ranked result(s) match are based on the user's data inputs from database 1a (structured data) and database 1b (structured and unstructured data) (as shown in FIG. 4) according to AI recommendations with the addition of NLP model processes; Level 2 best ranked results(s) are optional AI recommendations based on ranking from Level 1 with the addition of data query results from selected haptic biometric data from a user's wearable device(s). Haptic bio feedback can include heart rate, blood pressure, oxygen level, posture position, and location; and Level 3 best ranked result(s) are optional AI recommendations based on the ranking from Level 2 with the addition of data query results from enhanced haptics from wearable devices used during Immersive Reality (IR) events experiences-IR referring to one or a combination of AR (augmented reality), VR (virtual reality), and/or MR (mixed reality), the combination referred to as XR (extended reality). Enhanced haptics in Level 3 may include air bursts, scent, audio, voice, eye tracking and gesture recognition control.

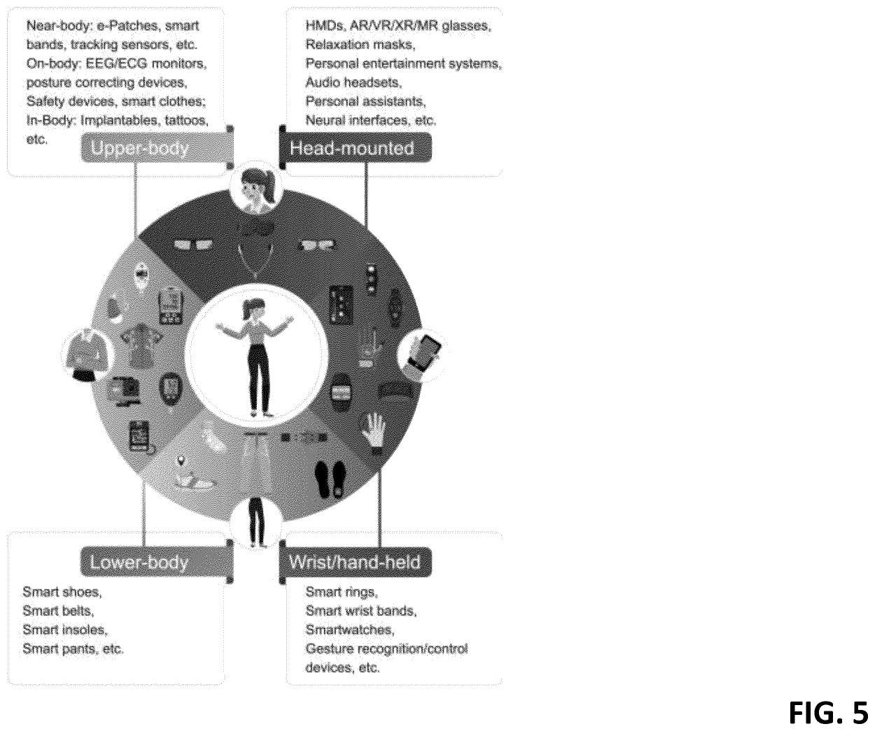

FIG. 5 (source: Ometov et al., Computer Networks 193 (2021) 108074, https://doi.org/10.1016/j.comnet.2021.108074) illustrates examples of possible measurement devices according to at least some embodiments as described herein. Some embodiments may include one, more than one, or all such possible measurement devices.

Second, at least some embodiments may personalize search and criteria parameters for each of the different types of user needs. Each user type (Jobseeker, Employer, or Educator) may rank and change the weighting of Level 1, Level 2, and Level 3 differently, and at different time periods, for different jobs and for different courses, culture & values to address the needs for the changing workforce of hybrid work and hybrid learning development.

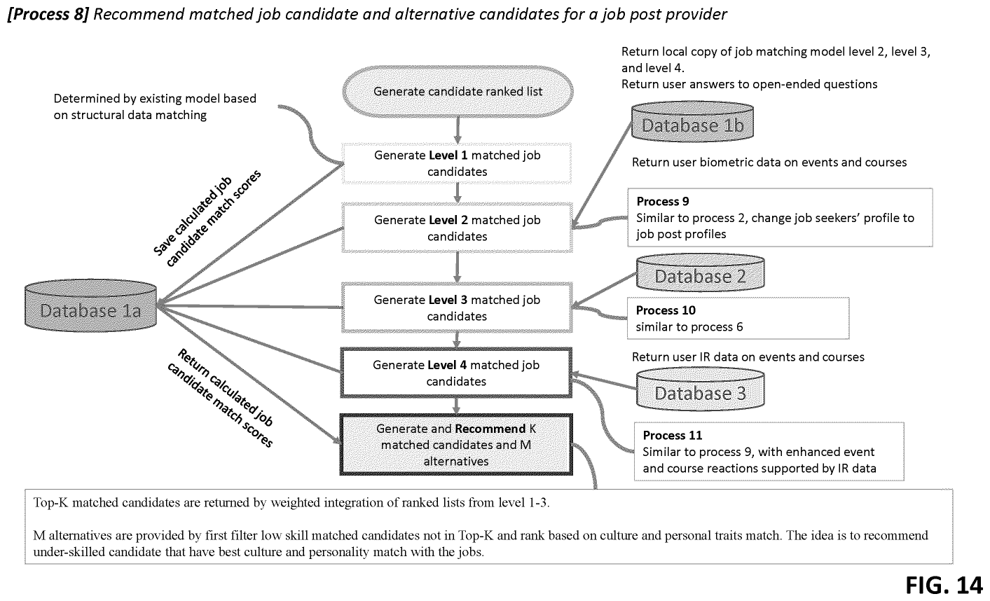

An illustration of a model for such an approach is illustrated in FIG. 14.

To explain the application of the model, the following non-limiting examples are provided for each of Jobseeker, Employer and Educator.

A Jobseeker (JS) completes his profile and gets a match to best fit jobs, and sees courses that pop up to help Jobseeker to attain those skills (Database 1 as shown in FIG. 4). This is Level 0. JS has the choice to rank weighting on data type inputs and so does for Level 1 as 90% and Level 2 at 10% (as JS has a fit bit and a smart watch) and Level 3 at 0% (until JS has attended immersive reality events). JS can change ranking when JS updates JS profile later.

Level 1 (Database 1b as shown in FIG. 4) takes into consideration JS interests through text longform answers, events and stories (video, podcasts, and/or written articles) that JS viewed on our SITE (as illustrated in FIG. 4), and stories that JS may have submitted to our site (NLP using both structured and non-structured data). Level 1 adds interests into the ranking formula. Interests results help to determine the type of activity and occupations and other industry sectors that may be of personal interest to JS. For example JS may have seen 2 job matches in results, but all in Oil and Gas. Level 1 provides addition job options for ESG instrumentation after JS viewed an article story on CO2 emissions and viewed a video story on an Entrepreneur in renewable energy on our SITE. AI nudges JS to look at 3 jobs in the EV (electrical vehicle) sector.

Level 2 (Databases 1 and 2 as shown in FIG. 4) takes into consideration real time activities from biometric feedback data from a wearable device on JS. The haptic feedback shows a raised heartrate and/or blood pressure (excitement) and/or oxygen levels during a marketing course event (education online and/or in-person). This is logged in JS user profile history. The AI may suggest additional job options to JS in the EV sector for client account manager and a marketing manager that may fit better with his motivation. The AI may also suggest communications courses to attain the communications & marketing skills needed in the predicted (inferred) job opportunities or career transition pathways (into another industry sector).

Level 3 (Database 1, 2, and 3 as shown in FIG. 4) takes into consideration real time activity reactions during Immersive Reality (IR) events (in this case a car racing gaming and a concert). The reactions during this event while he had on haptic gloves, google glasses and a haptic vest showed good coordination and dexterity and reaction time, and that JS interacted with other avatar teams in the car racing room and the bar in IR. This is logged in JS user history. The AI may suggest additional job options to JS: in this case an opportunity to apply as a manager in product development arm of Ferrari. If interested, JS hits the link on our SITE to that job where it says to apply first by participating in Ferrari's IR test portal. JS applies and receives a link to the IR portal for the cognitive test. JS also decides that he will view the suggested courses suggested by the AI to improve his chances of transitioning career pathways.

An Employer (EP) completes a job posting (skills, attributes and job profile description) and also sees the courses that match the skills EP is looking for in the candidate. EP gets matches to 2 best fit candidates that applied for the job, and to the 6 candidates (from other industry sectors) that viewed the job but did not apply. This is Level 0. EP has the choice to rank weighting on data type inputs and so does for Level 1 as 75%, Level 2 as 15% (as health and wellness are a factor) and Level 3 as 10% (as interests are key to core corporate culture). EP can change ranking of the Levels 1, 2, and 3 for each type of job posting.

Level 1 (Database 1b as shown in FIG. 4) takes into consideration EP job profile (EV Product Development) text longform answers on corporate culture, values, and goals, as well as job roles and activities (NLP using both structure and non-structured data). Level 1 adds this into the ranking formula. This information result helps to determine the type of attributes, transferable skills, and related activities that may also be a match to other candidates in other industry sectors. EP may have seen 2 best fit candidates in Level 0, but with the addition of Level 1 the AI nudges EP to look at 3 additional candidates that attended the CO2 emissions course, and who viewed the job, but did not apply. However EP had also weighted Level 2 query at 10% weighting.

Level 2 (Database 1a, 1b and 2 as shown in FIG. 4) takes into consideration real time activities from biometric feedback data from a wearable device on the 3 additional candidates. The AI indicates that 2 of the 3 additional candidates had biometric biofeedback history in their profiles. These candidates showed a reaction levels (a raised heart rate and oxygen levels) biofeedback interpretation to a local node (and then to the federated model cloud for process Level 2) during a marketing course event (education online and/or in person). The AI suggests that these additional candidates may be a fit from a motivation and interest perspective. The AI may also suggest that these 2 candidates also showed interest in taking communications and marketing courses to attain additional skills to align with a better fit to the job posting role needs.

Level 3 (Database 1a, 1b, 2, and 3 as shown in FIG. 4) takes into consideration real time activity reactions during Immersive Reality virtual events. The reactions from 2 additional candidates attended IR events (co-hosted on our SITE) for car racing event and a concert in IR. The 1 candidate (JS1) had a higher ranking for high emotional quotient than (JS2) and is ranked accordingly. The AI may recommend to EP that JS1 candidate when calculated with Level 0, 1, 2, and 3 has the core skills, attributes, motivation and capabilities for manager job posting in the product development department of Ferrari. EP responds after seeing the JS1 IR testing results for an interview.

An Educator (ED) completes a course posting for marketing, and sees many JSs and EPs viewed the post for a total of 10 views, but only 2 of the 10 views hit the link to the course posting on the ED site. This is Level 0. The course is offered as a hybrid option or as an in-person delivery. The challenge for ED is how to develop and position future offerings of this course to better align with the future shifting needs of both JS and EP in real time. EP has a challenge with student enrollment, engagement and content relevance to EPs. The current ways ED interacts with EPs is to meet a few times a year with EDs and as well through online ratings with JSs. ED seeks to redesign the content and mode of engagement and delivery to improve the alignment of shifting skills needs to agile course delivery, and as well the engagement and interaction of students during the courses in real time-throughout the year. This will help to reduce the costs of constant course redesign, and optimize the instructor fit and delivery channels.

An additional challenge for ED (and EP in small & medium sized organizations) is covered in Section C (below) on barriers to access due to operations costs, latency, interoperability and infrastructure gaps (with interest but not limited to Federated learning models for integrating WIOT into workforce development).

Level 1 (Database 1b as shown in FIG. 4) takes into consideration EP job profile (EV Product Development) text longform answers on corporate culture, values, and goals, as well as job roles and activities and the number of times that JS views the course, and the number of registrations to the course (NLP using both structure and non-structured data). Level 1 adds this into the ranking formula. This information result helps ED to also see how many times that this course appears alongside the skills posted.

Level 2 (Database 1 and 2 as shown in FIG. 4) takes into consideration real time activities from biometric feedback data from a wearable device on the 3 additional candidates. The AI indicates that 2 of these candidates showed a raised heartrate (excitement) and oxygen levels during a marketing course event (education online and/or in person). The AI may suggest that this form of course delivery and course content ranks higher than other marketing courses. The AI may also provide JS feedback on the course instructor. The AI may also nudge ED that JS also looked at communications courses for certain job posts where marketing was a skill. ED may decide to place a greater focus on communications courses to better align with the skills deliverables for JS and staff of ED.

Level 3 (Database 1, 2, and 3 as shown in FIG. 4) takes into consideration real time activity reactions during Immersive Reality virtual events. The reactions from the JS that viewed this marketing course also showed an interest in car racing and a high emotional reaction in how JS interacted with others. The AI may recommend to EP that this JS candidate may take the course if ED has an interactive or immersive reality component for a communications course or if the course has a case study related to gaming or sports events. ED may also look to develop an immersive reality test for EP to test JS candidate skills in a virtual environment as the next step of screening for the EV product development manager at Ferrari.

The following are non-limiting descriptions of our Multi-Mode Level 1 to Level 3 processes in an approach according to at least some embodiments as described herein: comprising of three types of users (Jobseekers, Employers, Educators) and Federated Learning Models. See also FIGS. 4 and 6 to 17, described herein as follows.

### Level 1: Predictive Recommendations by Enabling NLP on Current System (Database 1a as Shown in FIG. 4) to Pull User Viewing or Attendance History.

We may enhance the predictive matches in user results through queries with the addition of ML computer coding 205 to pull user history and profiles using NLP queries on data storage for stories (text, video, and/or audio) viewed, events (attendance or post recordings viewed such as our speaker series) attended or viewed (user history). In some embodiments, employers can find matching jobseeker candidates from another industry sector in addition to finding candidates in the employer's current industry sector that match the job profile. For example, some embodiments may provide workforce development to a jobseeker in his/her current industry sector, and predictive options (suggestions or nudges to self-navigate to other potential job opportunities) in different industry sectors. Some embodiments may match the industry sector that the jobseeker is currently working. Competitors use NLP matching algorithms for key word search from resumes or profiles. One example is LinkedIn where jobseekers are able to copy the skills (such as key words) required in the job posting into their online profile to game the system. Some embodiments such as those disclosed herein may overcome how JS games Linked-in with the addition of the latent skills & capabilities (biometrics and haptic from wearables and IR event experiences) and the courses that enable JS to gain those skills (results to the jobseeker) stated in the employer job posting. JS does not see the skills posted by the employer until JS is matched by our AI. Educators may post the skills gained from taking the course/training (badges and certifications are added in the course profile). Some embodiments such as those disclosed herein may match in another industry sector (a job posting that normally may not be presented by HRTech). For example, a restaurant waiter may be a match for several new jobs as a sous chef, but NLP ML models such as those described herein may query his long form answers (on the .ai data base 1b as shown in FIG. 4) mentioning food and health, the health topic events that he attended, and the videos on health topics viewed (user analytics history from .com) our predictive AI (ML model) may suggest to the waiter an additional job opportunity in another industry sector as a sous chef or dietician at a healthcare facility.

### Level 2: Biometric Data Enhancement—Additional Match Recommendations.

To enhance the results from Level 1, some embodiments such as those described herein may add another layer of user information in Level 2. Here some embodiments such as those described herein may add ML computer models to query biometric data from wearable devices. Some embodiments such as those described herein may involve the addition of biometric data for use in prescreening assessment and matching methodology (ML models) for workforce advancement using the combined queries from the 3 user group history and profiles. In some embodiments such as those described herein, the ML model learns from the queries from 3 user groups of jobseekers, educators, and employers and the addition of biometric data. For example, wearables such as smart watches, glasses, and/or wearable devices around the body may provide bio feedback on many levels. In some embodiments such as those described herein, the data collected from the wearable device 206 may use Tiny ML (edge processing at the wearable device level) to send data to the Fog Computing Node 209 for processing (which may further reduce latency) for the ML Data processing model in the Federated Cloud 208, which may query the data from the node (in real time internet-of-things (IoT) applications). ML queries (semantic layer) in a Federated Learning Model (e.g., Cloud ML Processing Model, data lake storage, data lake, data sources & infrastructure) in some embodiments as described herein may look for specific relevant data (from interpretation matrices for biometric feedback) results in profile and activity histories to add to the assessment and matching algorithms 203 and 204 and predictive ML models 205).

For example, to explain adding biometric data according to some embodiments as described herein, reference is made back to the waiter from the Level 1 process. The restaurant waiter is a match for several new jobs as a sous chef, and our NLP ML models query his long form answers mentioning food and health, the health topic events that he registered or viewed, and the videos on health topics viewed, now has biometric data from his smart watch indicates that he exercises more than the average person is now added to raise the ranking of this additional job opportunity as a sous chef or a dietician at a healthcare facility. The restaurant waiter looks at this opportunity as a sous chef or a dietician at a healthcare facility and sees that there are a few skills that he is not sure that he has. The courses that are matched to each different job opportunity can deliver the different skills needed. The biometric data from his smart watch also tracks stress and health & wellness indicators like high heart rate and high blood pressure. The number of hours and shift work needed to be a sous chef are stressful. The hours for a dietician are more regular. The dietician job gets ranked higher from this perspective; however the waiter will need to decide from the information presented on the costs and time to upskill to a dietician job vs to a sous chef as the best choices for him to follow through.

Another example is how some embodiments such as those described herein can use the biometric data from virtual and hybrid learning events (to advance career development) from the perspectives of employers or educators as users. Educator may seek to improve student engagement through gamification of virtual learning events. She may have her students use a wearable device to interact with a desktop screen or a mobile device to answer questions. For example the use of a QR code or disposable skin patches to activate the entry to a game situation or a video for a case study in Immersive Reality (IR) for class groups to solve together or individually. Any one or all of the heart rate, arm movement, head movement or location bio feedback can be collected, along with the answers and reactions to the case problem that interpret (matrix interpretation models), the engagement level by the students during the live presentation, and as well the timing of reactions (WIOT signals even when a student's camera is not on) during the class course or workshop. The biofeedback results can be further enhanced in Stage 3. Employer may be searching for a jobseeker's cultural fit and alignment with his company's values. If the job requirements need to be in a physically demanding outdoor job, the rankings of candidates may be raised or lowered based on biofeedback related to amount of physical activity and time spent hiking (steps) location and heart rate or other haptic feedback (e.g. Blood Pressure, O2 level, respiratory level, and/or skin temperature) may play a role in ranking of the candidates (if candidate data is available) for job activity match ranking. In some embodiments, Employer and Educator users do not see this level of personal data (privacy) as it may be managed by processes (Users see feedback through the interpretation matrices) that are not accessible to the Employer or Educator. A ranking may recommend candidates that best match—as an example: outdoors work and higher physical activity tolerance. This ranking can be further enhanced with the addition of Level 3 Assessments.

### Level 3: Immersive Reality Experience Enhancement-Additional Match Recommendations.

To enhance the result from Stage 2, some embodiments such as those described herein may add experiences from participation in events through Immersive Reality (IR). The devices used in IR can include Virtual Reality (VR), Augmented Reality (AR), Mixed Reality (MR), and Extended Reality (XR). In some embodiments, the event is experienced through more than just audio and visual. The event could be a wedding, a concert, a sports event, a game, leisure of lifestyle activities, or even a course or workshop where training is involved, or a manufacturing process where a product is made. Additional reactions through haptic feedback can be collected in this environment in Level 2, as the devices may provide a higher level of environmental haptic feedback through event experiences such as those found in games. For example wearables that enable motion sensing, scent, audio, and/or air blasts may be used.

In some embodiments such as those described herein, the data collected from the wearable device may use Tiny ML (edge processing at the wearable AV/AR/MR device level 207) to send data to the Fog Computing Node 210 for processing (further reducing latency) for the ML Data processing model in the Federated Cloud 208, which may query the data from the node (in real time IoT applications). ML queries in a Federated ML Cloud (e.g., ML Processing Model, data lake storage & data lake) of some embodiments such as those described herein may look for specific data of relevance from the biometric feedback (for our assessments and matching algorithms 203 and 204 and ML models 205) to generate, predict, and recommend additional options to the user.

For example, to explain the application for Jobseekers in some embodiments, if the person has attended one or several types of events, the type of event and the level of haptic feedback may enable the ML model to suggest additional options for a career advancement or a learning advancement pathway.

For example, to rank the job candidates for Employers, the type and number of such events and the positive or negative aspects of this biofeedback may indicate the types of environmental preferences, stimuli, and personal hobbies that may align or not with the company's work environment or culture. The candidate ranking may be adjusted to the employer's preferences for such attributes in addition to the skills required for the job.

For example, to improve the alignment, content, and course delivery of an educator's class, or program content or mode of delivery, the engagement level and number of reactions, questions, and/or answers from a student may be important for the educator. A class could problem solve a case in a gamified portion of the class by teams, and then discuss in the general class how each team they approached their problem and solved it. The biofeedback and haptic feedback reactions of the experiences can be collected during the game through the wearable devices and following the gamified portion during the live (e.g. zoom) discussion can be collected. Devices can be disposable or reusable patches and or devices worn on or around the body.

Third, some embodiments such as those described herein may optimize systems latency and operations costs to advance the interoperability of Cloud Systems and Wearable Devices in Virtual environment—as a means to provide affordable, effective access to Workforce Development (employability and education) for SMES. See FIGS. 4 and 6 to 11 and the definitions below.

In this approach, some embodiments such as those described herein may reduce the high cost and knowledge intensive barriers to entry to afford and navigate the systems and infrastructure required to play in the machine learning field. Educational institutes and organizations are just now learning how to design, develop and deploy virtual online classes. What is truly lacking as evidenced by both students and instructors, is an accessible and affordable way to engage the participants in quality learning. Large corporations are adopting internal training and talent redeployment as a way to address the skills gap and have the resources to access the newest technologies or build their own. However, SMEs who drive the economy are still waiting in the hallways.

Some embodiments such as those described herein may address one, more than one, or all of the following.

MLOPs Architecture Approach: Educator Classes and Employer Remote Work Engagement-Fog Nodes for schools and homes (see FIG. 4); Students and Employees-Tiny ML-Device parameters for real time feedback and to reduce the load of compute data going to the global cloud; Affordability and Cost Access Cloud architecture for MLOPs Cost and Delivery—Focus to Federated Learning Models for Wearable Internet of Things (WIOT); ESG—lower footprint & energy consumption from a reduced computer load in the global cloud and in local parameter clouds.

Some embodiments such as those described herein may reduce the type and amount of centralized data on the global cloud, with a copy of the data on the local cloud.

Some embodiments such as those described herein may reduce the latency of the global cloud and speeds up the performance response to users

Some embodiments such as those described herein may reduce the risk of security on the global cloud by storing only the results and histories, and a copy is kept on the parameter cloud (related to each of Database 1a, 1b, 2, and 3 as shown in FIG. 4).

Some embodiments such as those described herein may improve the interoperability of multi-cloud, multi-database systems. Some embodiments such as those described herein may reduce the security risk of a single global cloud by limiting access to specific layers to specific databases in specific clouds

Semantic layer: a semantic layer according to some embodiments such as those described herein may query the results and histories of the users in the data lake-pulling out the supervised ML ranking results of Level 0 to 3 assessments. Some embodiments such as those described herein may provide greater security by storing levels of data on different clouds, and an API that activates activity to reduce computation power and system latency when there is a query for specific data

Wearables (WIoT): some embodiments such as those described herein may use fog node and tiny ML in devices (multi-access edge computing) to reduce the computational power and time required to process results. The interpretation and ranking of results may, in some embodiments, be processed in Level 3 assessments only to reduce to operating costs of computing and storage of data.

### Definitions

### Federated ML Learning (FL):

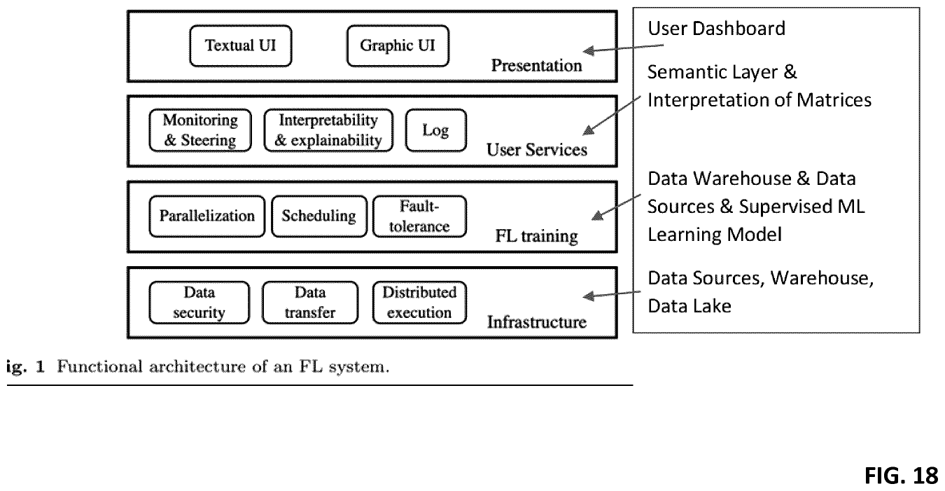

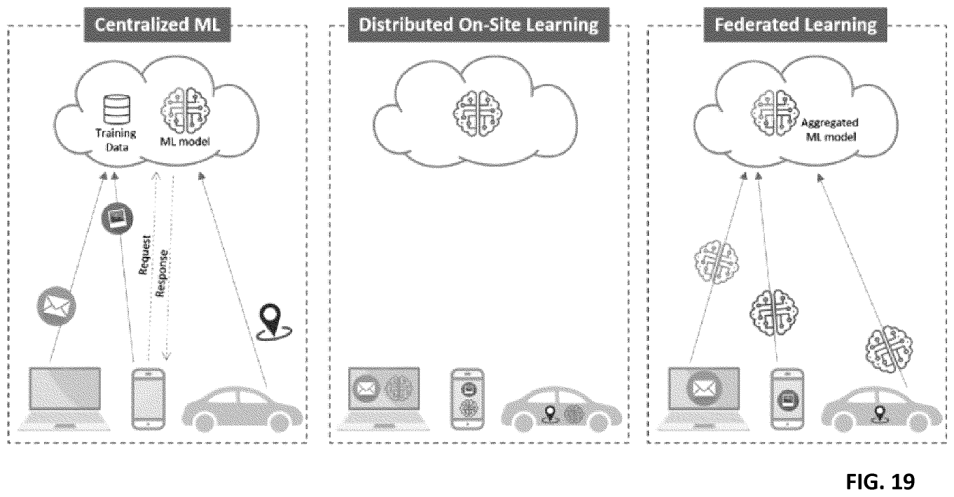

See FIG. 18 for an architecture example (source: Ji Liu‡ et al Baidu Inc., Beijing, China). See FIG. 19 for an illustration of distributed vs federated FL (source: (PDF) A Survey on Federated Learning: The Journey From Centralized to Distributed On-Site Learning and Beyond (researchgathe.net) (https://www.researchgate.net/publication/344871928_A_Survey_on_Federated_Learning_The_Journey_From_Centralized_to_Distribute_On-Site_Learning_and_Beyond)). Some embodiments such as those described herein may involve a Federated learning model (FL), where the data processing can be done on the “local” CPU/GPU and copy of history saved to “local “parameter cloud and results sent to data lake in FL. Semantic layer pulls history from several parameter clouds and matches and ranks results and sends recommendations back to local CPU/data processor to send results back to users. Users meaning Jobseekers, Employers & Educators.

The traditional AI algorithms require centralising data on a single machine or a server. The limitation of this approach is that all the data collected is sent back to the central server for processing before sending it back to the devices. The whole process limits a model's ability to learn in real-time (source: https://analyticsindiamag.com/distributed-machine-learning-vs-federated-learning-which-is-better/).

Federated Learning is a centralised server first approach. It is a distributed ML approach where multiple users collaboratively train a model. The concept of federated learning was first introduced in Google AI's 2017 blog (https://ai.googleblog.com/2017/04/federated-learning-collaborative.html). Here, the raw data may be distributed without being moved to a single server or data centre. It selects a few nodes and sends the initialised version containing model parameters of an ML model to all the nodes. Each node may execute the model, may train the model on their local data, and may have a local version of the model at each node.

Federated Learning leverages techniques from multiple research areas such as distributed systems, machine learning, and privacy. FL is best applied in situations where the on-device data is more relevant than the data that exists on servers.

Federated learning may provide edge devices with state of the art ML without centralising the data and privacy by default. Thus it may handle the unbalanced and non-Independent and Identically Distributed (IID) data of the features in mobile devices. A lot of data may be generated from smartphones that can be used locally at the edge with on-device inference. Since the server does not need to be in the loop for every interaction with the locally generated data, this enables fast working with battery saving and better data privacy.

### Semantic Layer

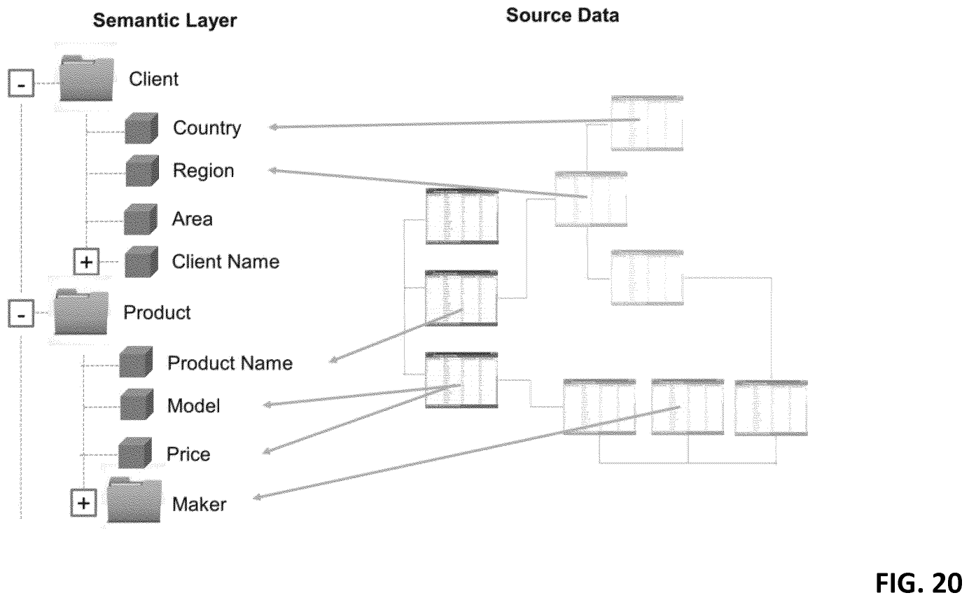

The semantic layer is just a representation of the data, it is a metadata layer—it does not contain any data. The Semantic layer contains information about the objects in the data source which it uses to generate queries to retrieve the data. So the Semantic layer allows to solve the issue with data meaning ambiguity. See FIG. 20 (source: Why do you need a semantic layer for your Data Lakes, Fabrizio Andreai, Mar. 12, 2019).

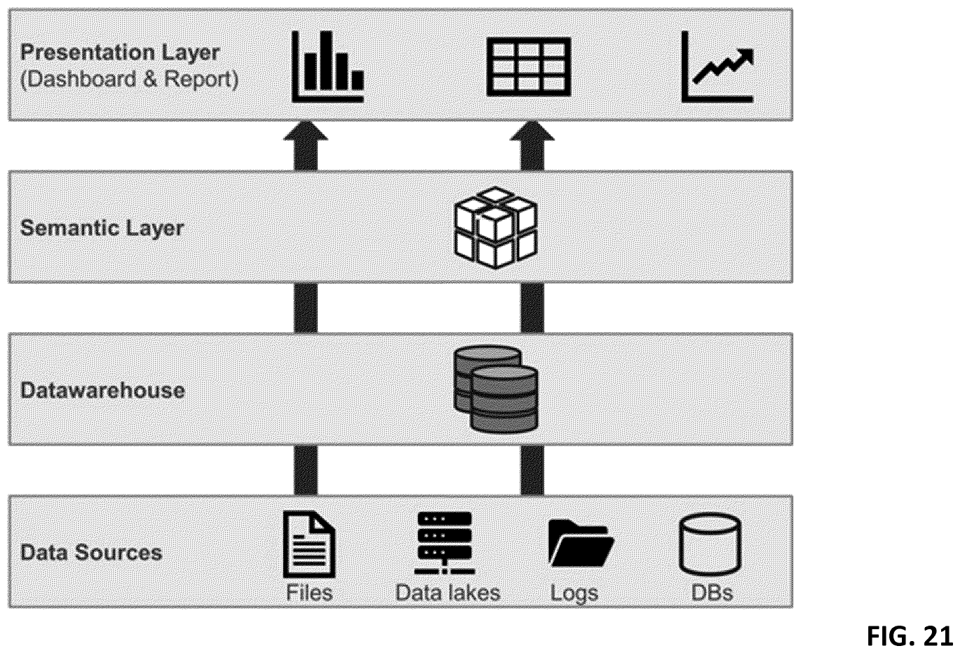

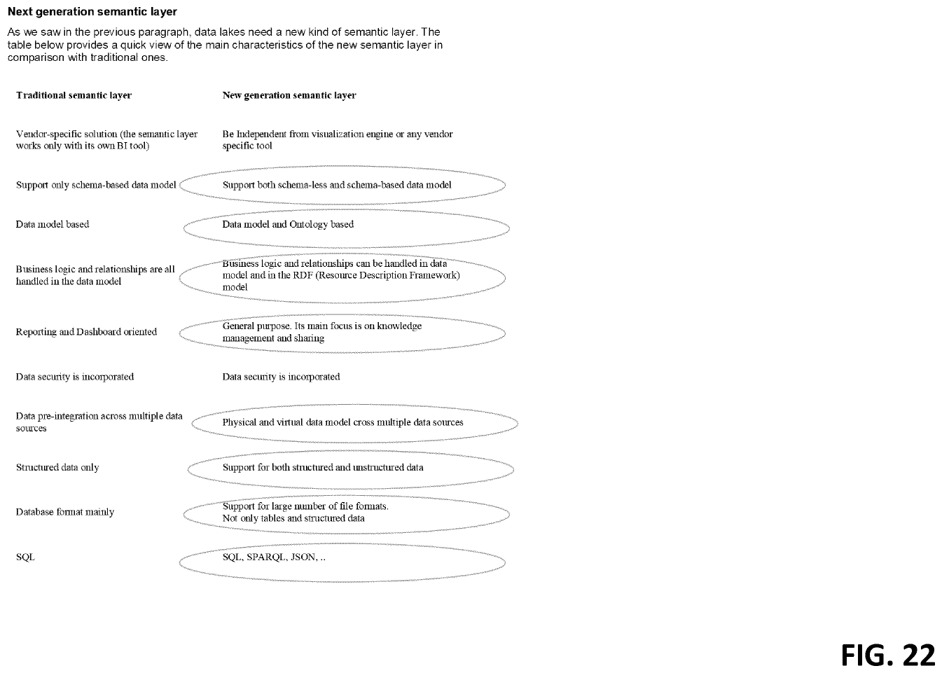

Traditionally, these semantic layers sit on top of a traditional data warehouse or databases in order to make easier for business to create their reports. Semantic layers may be the main entry points for data access for most business users when they are creating reports, dashboards, or running ad hoc queries. They have generally been purpose-built for specific BI visualization tools and generally are vendor-dependent. See FIGS. 21 and 22 (source: Why do you need a semantic layer for your Data Lakes, Fabrizio Andreai, Mar. 12, 2019).

Source Note IBM: Ontology-based recommender systems may exploit hierarchical organizations of users and items to enhance browsing, recommendation, and profile construction. This transverse evaluation may provide insights into the utility of different information resources and methods for the initial stages of recommender system development.

Citations and Reference Sources from Preliminary Research

Deep learning approaches are machine learning methods used in many application fields today. Some core mathematical operations performed in deep learning are suitable to be parallelized. Parallel processing increases the operating speed. Graphical Processing Units (GPU) are used frequently for parallel processing. Parallelization capacities of GPUs are higher than CPUs, because GPUs have far more cores than Central Processing Units (CPUs) (source: https://ieeexplore.ieee.org/document/8751930).

Central processing units (CPUs) may be composed of millions upon millions of tiny transistors with multiple “cores.” They may be critical for handling the main processing functions of some computers. Actions like running the operating system and applications may not be possible without it. The CPU is also what may determine the general speed of a computer (source: https://www.incredibuild.com/blog/cpu-vs-gpu-know-the-difference).

Even though the central part of data processing may take place in different computing layers, sometimes minor filtering processes may be executed in wearable and IoT devices. The early pre-processing stage may be important for wearable and IoT devices since they may not have enough computational and storage resources to process “unnecessary” data.

See also paragraphs 3.3.4 to 4.9 from Ometov et al., Computer Networks 193 (2021) 108074, which contain subject matter that may be useful, but are not intended to be limiting in any way.

### General Discussion of Some Disclosed Embodiments

In general, embodiments such as those described herein may involve identify at least one opportunity for personal development of an individual in response to an inference, from at least one measurement of a body of the individual, of at least one cognitive reaction of the individual.

Such an inference may be identified as described in Dzedzickis et al., Sensors 2020, 20, 592; doi:10.3390/s20030592, for example.

In any context in this disclosure, a cognitive reaction of an individual may be an emotion of the individual, or may be another cognitive reaction of the individual.

In any context in this disclosure, a cognitive reaction of an individual may indicate, for example, career aptitude of the individual, a personal value of the individual, a personal goal of the individual, a cultural fit of the individual, a hobby of the individual, or a combination of two or more thereof.

In any context in this disclosure, personal development may include, but is not limited to, knowledge and experience advancement in career, education, or skill, health & wellness development, or leisure or hobby development, or a combination of two or more thereof.

In any context in this disclosure, opportunities may include, for example, best ranked result(s) as described above, suggested careers, skills, courses, career candidates, or events as described above, matching or matches as described above, other options or opportunities as described above, suggested candidates as described above, courses as described above, or still other opportunities including but not limited to opportunities as described above.

In any context in this disclosure, opportunities for personal development may include one or more educational opportunities, for example opportunities for education that may benefit an individual according to at least one measurement of a body of the individual. Such an educational opportunity may be advertising-related.

Additionally or alternatively, in any context in this disclosure, opportunities for personal development may include one or more advertising-related opportunities, for example advertising that may be of interest to an individual according to at least one measurement of a body of the individual.

Additionally or alternatively, in any context in this disclosure, opportunities for personal development may include one or more career opportunities, for example careers that may be suitable for an individual according to at least one measurement of a body of the individual.

Additionally or alternatively, in any context in this disclosure, opportunities for personal development may include one or more career-development opportunities, for example career development that may be suitable for an individual according to at least one measurement of a body of the individual. Such a career-development opportunity may be advertising-related.

Additionally or alternatively, in any context in this disclosure, opportunities for personal development may include one or more career-development opportunities and one or more educational opportunities. Such one or more career-development opportunities and one or more educational opportunities may be presented separately or simultaneously.

Additionally or alternatively, in any context in this disclosure, opportunities for personal development may include one or more entertainment opportunities. An entertainment opportunity may include a food-related opportunity, a sport-related opportunity, an arts-related opportunity, a music-related opportunity, a travel-related opportunity, or a combination of two or more thereof. Such an entertainment-development opportunity may be advertising-related.

In general, one or more indicators of a career-development opportunity of an individual may include career aptitude of the individual, a personal value of the individual, a personal goal of the individual, a cultural fit of the individual, a hobby of the individual, or a combination of two or more thereof.

In any context in this disclosure, cognitive reactions may be identified using methods based on an unconscious response of electrical parameters measured using one or more sensors.

In any context in this disclosure, cognitive reactions may include excitement, interest, happiness, anger, stress, disgust, disinterest, boredom, sadness, or a combination of two or more thereof.

Weighting, as indicated above for example, may allow an individual to personalize any method such as one or more of the methods described herein.

### CLAUSES

This disclosure includes but is not limited to the following clauses, which may be combined with other subject matter in this specification.

1. A method of facilitating personal development of an individual, the method comprising causing at least one processor circuit to, at least, in response to an inference, from at least one measurement of a body of the individual, of at least one cognitive reaction of the individual, identify at least one opportunity for personal development of the individual.

2 The method of clause 1 further comprising causing the at least one processor circuit to, at least, infer, from at least one measurement of a body of the individual, an inference of at least one cognitive reaction of the individual.

3 The method of clause 2 wherein:
causing the at least one processor circuit to infer the at least one cognitive reaction comprises causing a first at least one processor circuit to infer the at least one cognitive reaction;
causing the at least one processor circuit to identify the at least one opportunity comprises causing a second at least one processor circuit to identify the at least one opportunity; and
the first at least one processor circuit is separate from and in network communication with the second at least one processor circuit.

4. The method of clause 3 wherein the first at least one processor circuit is at least one processor circuit of an edge device.

5. The method of clause 3 wherein the first at least one processor circuit is at least one processor circuit of a fog node.

6. The method of clause 3, 4, or 5 wherein the second at least one processor circuit is at least one processor circuit of a server computer distinct from the first at least one processor circuit.

7 The method of clause 3, 4, 5, or 6 wherein the first at least one processor circuit is in wireless network communication with the second at least one processor circuit.

8. The method of any one of clauses 3 to 7 wherein the first at least one processor circuit is in Internet communication with the second at least one processor circuit.

9. The method of any one of clauses 1 to 21 further comprising causing the at least one processor circuit to, at least, receive, from at least one measurement device, at least one signal indicating the at least one measurement.

10. The method of clause 9 further comprising causing the at least one processor circuit to, at least, cause the at least one measurement device to measure the at least one measurement.

11. The method of clause 9 or 10 wherein the at least one measurement device is worn on the body of the individual when the at least one measurement device measures the at least one measurement.

12. The method of clause 9, 10, or 11 wherein the at least one measurement device comprises at least one haptic measurement device.

13. The method of any one of clauses 1 to 12 wherein the at least one measurement comprises at least one haptic measurement of the body of the individual.

14. The method of any one of clauses 1 to 13 wherein the at least one measurement comprises at least one biometric measurement of the body of the individual.

15. A system for facilitating personal development of an individual, the system comprising:
at least one processor circuit; and
at least one computer-readable storage medium comprising stored thereon program codes that, when executed by the at least one processor circuit, cause the at least one processor circuit to, at least, in response to an inference, from at least one measurement of a body of the individual, of at least one cognitive reaction of the individual, identify at least one opportunity for personal development of the individual.

16. The system of clause 15 wherein the program codes, when executed by the at least one processor circuit, further cause the at least one processor circuit to, at least, infer, from at least one measurement of a body of the individual, an inference of at least one cognitive reaction of the individual.

17. The system of clause 16 wherein:
causing the at least one processor circuit to infer the at least one cognitive reaction comprises causing a first at least one processor circuit to infer the at least one cognitive reaction;
causing the at least one processor circuit to identify the at least one opportunity comprises causing a second at least one processor circuit to identify the at least one opportunity; and
the first at least one processor circuit is separate from and in network communication with the second at least one processor circuit.

18. The system of clause 17 wherein the first at least one processor circuit is at least one processor circuit of an edge device.

19. The system of clause 17 wherein the first at least one processor circuit is at least one processor circuit of a fog node.

20. The system of clause 17, 18, or 19 wherein the second at least one processor circuit is at least one processor circuit of a server computer distinct from the first at least one processor circuit.

21. The system of clause 17, 18, 19, or 20 wherein the first at least one processor circuit is in wireless network communication with the second at least one processor circuit.

22. The system of clause 17, 18, 19, or 20 wherein the first at least one processor circuit is configured to be in wireless network communication with the second at least one processor circuit.

23. The system of any one of clauses 17 to 22 wherein the first at least one processor circuit is in Internet communication with the second at least one processor circuit.

24. The system of any one of clauses 17 to 22 wherein the first at least one processor circuit is configured to be in Internet communication with the second at least one processor circuit.

25. The system of any one of clauses 15 to 24 wherein the program codes, when executed by the at least one processor circuit, further cause the at least one processor circuit to, at least, receive, from at least one measurement device, at least one signal indicating the at least one measurement.

26. The system of clause 25 wherein the program codes, when executed by the at least one processor circuit, further cause the at least one processor circuit to, at least, cause the at least one measurement device to measure the at least one measurement.

27. The system of clause 25 or 26 further comprising the at least one measurement device.

28. The system of clause 27 wherein the at least one measurement device is wearable on the body of the individual when the at least one measurement device measures the at least one measurement.

29. The system of clause 27 or 28 wherein the at least one measurement device comprises at least one haptic measurement device.

30. The system of any one of clauses 15 to 29 wherein the at least one measurement comprises at least one haptic measurement of the body of the individual.

31. The system of any one of clauses 15 to 30 wherein the at least one measurement comprises at least one biometric measurement of the body of the individual.

### CONCLUSION

In general, skill gaps may be evolving as soft skills (such as writing and speaking) join the categories of hard skills (such as programming skills) as companies adopt AI, adapt to automation, and integrate hybrid work and remote work. Due to a shift to AI and automation, there may be a critical unmet economic need globally for skilled workers. For example, there may be 85 million unfilled jobs (https://www.kornferry.com/insights/this-week-in-leadership/talent-crunch-future-of-work) in the United States, and a potential $1.75 trillion GDP gain for the US economy if skill gaps can be bridged (Korn Ferry 2022). In Canada, there may be a potential $50 billion GDP gain for the Canadian economy if skill gaps can be bridged (Deloitte 2022). Businesses with less than 500 staff may be increasingly impacted by recruitment and retention, time and costs. For example, in the US (and North America), 52% of small and medium sized businesses with less than 500 staff may not be able to find qualified new hires (CNBC 2022), and new hires may, on average, leave after 30 days (referred to as churn rate). This critical unmet economic need may benefit from better approaches to improve productivity of small and medium sized businesses. Such approaches may include, for example, automating prescreening processes to save employers time and overhead costs, prequalifying talent by matching employer job post skill needs to training and course posts, providing employer skill demands in real-time info to enable educators to improve the alignment of course content and delivery to industry skill demands, and upskilling, reskilling, and retraining talent to match to each specific job posting with real-time info and to reduce the amount of churn.

Recruitment and retention may be key challenges for all employers. With a rise in AI adoption, hybrid work, and remote work, employers are starting to consider a match of candidate values to company culture as a more critical element of productivity. Values (e.g., that arise from personality assessments for cultural, situational and behavior) of changing demographics add to the complexity. Assessments for personality values may not be noted in a job post, as such assessments are usually done during labour intensive in-person interviews. Existing automated applicant tracking systems may not collect information needed to assess and match candidate values to company culture. Such systems may track for skills, but may not provide ways to assess personality, interests, or preferences. Further, existing matching platforms may rely on keyword parsing, which may result in missed or mismatched skills.

Embodiments such as those described above may match a job applicant or student with opportunities such as job postings, learning opportunities, or both without requiring human intermediaries or intermediaries other than embodiments such as those described above. For example, according to embodiments such as those described above, job applicants and students may be matched with opportunities without requiring human-resource consultants, recruiters, educational consultants, or other intermediaries. Further, embodiments such as those described above may use semantic matching approaches to match job applicants and students with opportunities based on applicant/student skills, personality characteristics, or both skills and personality characteristics.

Although specific embodiments have been described and illustrated, such embodiments should be considered illustrative only and not as limiting the invention as construed according to the accompanying claims.

1. A method of facilitating personal development of an individual, the method comprising causing at least one processor circuit to, at least, in response to a match between the individual and at least one opportunity, the at least one opportunity withheld from the individual prior to the match, present the at least one opportunity to the individual.

2. The method of claim 1 further comprising causing the at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

3. A method of matching an individual to at least one opportunity, the method comprising causing at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

4-6. (canceled)

7. The method of claim 2 wherein the at least one opportunity comprises at least one learning opportunity.

8. (canceled)

9. The method of claim 2 wherein the at least one opportunity comprises at least one employment opportunity and at least one learning opportunity providing knowledge associated with the at least one employment opportunity.

10. (canceled)

11. The method of claim 2 wherein the inference is further based on at least one measurement of a body of the individual.

12. The method of claim 11 further comprising causing the at least one processor circuit to, at least, receive, from at least one measurement device, at least one signal representing the at least one measurement.

13. (canceled)

14. The method of claim 12 wherein the at least one measurement device is worn on the body of the individual when the at least one measurement device measures the at least one measurement.

15-25. (canceled)

26. The method of claim 11 wherein the at least one measurement comprises at least one haptic measurement of the body of the individual.

27. The method of claim 11 wherein the at least one measurement comprises at least one biometric measurement of the body of the individual.

28-70. (canceled)

71. A system for facilitating personal development of an individual, the system comprising:
at least one processor circuit; and
at least one computer-readable storage medium comprising stored thereon program codes that, when executed by the at least one processor circuit, cause the at least one processor circuit to, at least, in response to a match between the individual and at least one opportunity, the at least one opportunity withheld from the individual prior to the match, present the at least one opportunity to the individual.

72. The system of claim 71 wherein the program codes, when executed by the at least one processor circuit, further cause the at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

73. A system for matching an individual to at least one opportunity, the system comprising:
at least one processor circuit; and
at least one computer-readable storage medium comprising stored thereon program codes that, when executed by the at least one processor circuit, cause the at least one processor circuit to, at least, in response to at least one inference of at least one personality characteristic of the individual, the at least one inference based on at least one response of the individual to at least one prompt, match the individual to the at least one opportunity based at least on the inferred at least one personality characteristic of the individual.

74-76. (canceled)

77. The system of claim 72 wherein the at least one opportunity comprises at least one learning opportunity.

78. (canceled)

79. The system of claim 72 wherein the at least one opportunity comprises at least one employment opportunity and at least one learning opportunity providing knowledge associated with the at least one employment opportunity.

80. (canceled)

81. The system of claim 72 wherein the inference is further based on at least one measurement of a body of the individual.

82. The system of claim 81 wherein the program codes, when executed by the at least one processor circuit, further cause the at least one processor circuit to, at least, receive, from at least one measurement device, at least one signal representing the at least one measurement.

83. (canceled)

84. The system of claim 82 further comprising the at least one measurement device, wherein the at least one measurement device is wearable on the body of the individual when the at least one measurement device measures the at least one measurement.

85-96. (canceled)

97. The system of claim 81 wherein the at least one measurement comprises at least one haptic measurement of the body of the individual.

98. The system of claim 81 wherein the at least one measurement comprises at least one biometric measurement of the body of the individual.

99-141. (canceled)

142. The method of claim 9 wherein the at least one learning opportunity provides knowledge required for the at least one employment opportunity.
